(19) United States
(12) Patent Application Publication
(10) Pub. No.: US 2025/0120733 A1
(43) Pub. Date: Apr. 17, 2025

(54) JAW CONTROL SURFACES ON A SURGICAL INSTRUMENT JAW

(71) Applicant: Cilag GmbH International, Zug (CH)

(72) Inventors: Frederick E. Shelton, IV, Hillsboro, OH (US); Jason L. Harris, Lebanon, OH (US); Adam D. Hensel, Gahanna, OH (US)

(21) Appl. No.: 18/379,826

(22) Filed: Oct. 13, 2023

(51) Int. Cl. A61B 17/29

(52) U.S. Cl. CPC A61B 17/29; A61B 2017/2944

(57) ABSTRACT

A surgical instrument comprising a first jaw and a second jaw rotatable relative to the first jaw is disclosed. The surgical instrument further comprises a translatable closure driver configured to engage the second jaw and move the second jaw into a closed position. The second jaw comprises control surfaces which interface with the closure drive to control and/or limit the movement of the second jaw relative to the first jaw.

### BACKGROUND

A surgical instrument is inserted to a patient during a surgical procedure and clamped onto patient tissue. The surgical instrument can include one or more jaws that are controllable by the surgical instrument.

### SUMMARY

In accordance with the present disclosure, a surgical instrument comprises a movable jaw that is movable from an open position to a closed position to clamp patient tissue against another jaw of the surgical instrument. The movable jaw comprises control surfaces that interface with a closure system of the surgical instrument when the jaw is closed. The movable jaw also comprises another set of control surfaces that interface with the closure system when the jaw is opened.

### LISTING OF THE FIGURES

Various features of the embodiments described herein, together with advantages thereof, may be understood in accordance with the following description taken in conjunction with the accompanying drawings as follows:

FIG. 1 is a perspective view of a surgical stapling instrument in accordance with the present disclosure;

FIG. 2 is a partial perspective view of the stapling instrument of FIG. 1;

FIG. 3 is a perspective view of an anvil of the stapling instrument of FIG. 1;

FIG. 4 is a partial plan view of the anvil of FIG. 3;

FIG. 5 is a partial elevational view of the anvil of FIG. 3;

FIG. 6 is a partial elevational view of the anvil of FIG. 3;

FIG. 7 is a partial plan view of the stapling instrument of FIG. 1 illustrated in an open, unclamped configuration;

FIG. 8 is a partial elevational view of the stapling instrument of FIG. 1 illustrated in the open, unclamped configuration of FIG. 7;

FIG. 9 is a cross-sectional view of the stapling instrument of FIG. 1 taken along 9-9 in FIG. 8;

FIG. 10 is a partial plan view of the stapling instrument of FIG. 1 illustrated in a closed, clamped configuration;

FIG. 11 is a partial elevational view of the stapling instrument of FIG. 1 illustrated in the closed, clamped configuration of FIG. 10; and

FIG. 12 is a cross-sectional view of the stapling instrument of FIG. 1 taken along 12-12 in FIG. 11;

FIG. 13 is an elevational view of a surgical end effector comprising an anvil shown in phantom lines in an open position in accordance with the present disclosure;

FIG. 14 is a cross-sectional end view of the surgical end effector of FIG. 13;

FIG. 15 is a partial cross-sectional perspective view of the anvil of the end effector of FIG. 13;

FIG. 16 is a side elevational view of an anvil closure member of the surgical end effector of FIG. 13;

FIG. 17 is a side elevational view of a firing member of the surgical end effector of FIG. 13;

FIG. 18 is a partial side elevational view of the surgical end effector of FIG. 13 with the anvil shown in an open position in phantom lines;

FIG. 19 is another partial side elevational view of the surgical end effector of FIG. 13 with the anvil shown in a closed position;

FIG. 20 is a side elevational view of the surgical end effector of FIG. 13 with the firing member at the beginning of a staple firing stroke; and

FIG. 21 is a side elevational view of the surgical end effector of FIG. 13 with the anvil closure member and the firing member being partially distally deployed in the end effector.

Corresponding reference characters indicate corresponding parts throughout the several views.

### DETAILED DESCRIPTION

Applicant of the present application owns the following U.S. Patent Applications that were filed on even date herewith and which are each herein incorporated by reference in their respective entireties:
U.S. Patent Application, titled METHOD OF OPERATING A SURGICAL STAPLING INSTRUMENT; Attorney Docket No. END9484USNP2/220491-2M;
U.S. Patent Application, titled SURGICAL STAPLING SYSTEMS WITH ADAPTIVE STAPLE FIRING ALGORITHMS; Attorney Docket No. END9484USNP3/220491-3;
U.S. Patent Application, titled LEARNED TRIGGERS FOR ADAPTIVE CONTROL OF SURGICAL STAPLING SYSTEMS; Attorney Docket No. END9484USNP4/220491-4;
U.S. Patent Application, titled CONTROL CIRCUIT FOR ACTUATING MOTORIZED FUNCTION OF SURGICAL STAPLING INSTRUMENT UTILIZING INERTIAL DRIVE TRAIN PROPERTIES; Attorney Docket No. END9484USNP5/220491-5;
U.S. Patent Application, titled PROPORTIONATE BALANCING OF THE FUNCTION IMPACT MAGNITUDE OF BATTERY OUTPUT TO PEAK MOTOR CURRENT; Attorney Docket No. END9484USNP6/220491-6;
U.S. Patent Application, titled MOTOR OPTIMIZATION BY MINIMIZATION OF PARASITIC LOSSES AND TUNING MOTOR DRIVE CONFIGURATION; Attorney Docket No. END9484USNP7/220491-7;
U.S. Patent Application, titled APPARATUS AND METHOD TO REDUCE PARASITIC LOSSES OF THE ELECTRICAL SYSTEM OF A SURGICAL INSTRUMENT; Attorney Docket No. END9484USNP8/220491-8;
U.S. Patent Application, titled SURGICAL TOOL WITH RELAXED FLEX CIRCUIT ARTICULATION; Attorney Docket No. END9484USNP9/220491-9;
U.S. Patent Application, titled WIRING HARNESS FOR SMART STAPLER WITH MULTI AXIS ARTICULATION; Attorney Docket No. END9484USNP10/220491-10;
U.S. Patent Application, titled SURGICAL SYSTEM WITH WIRELESS ARRAY FOR POWER AND DATA TRANSFER; Attorney Docket No. END9484USNP11/220491-11; and.

U.S. Patent Application, titled SURGICAL STAPLE CARTRIDGE COMPRISING REPLACEABLE ELECTRONICS PACKAGE; Attorney Docket No. END9484USNP12/220491-12.

Applicant of the present application owns the following U.S. Patent Applications that were filed on even date herewith and which are each herein incorporated by reference in their respective entireties:
U.S. Patent Application, titled METHOD OF ASSEMBLING A STAPLE CARTRIDGE; Attorney Docket No. END9484USNP13/220491-13M;
U.S. Patent Application, titled CONTROL SURFACES ON A STAPLE DRIVER OF A SURGICAL STAPLE CARTRIDGE; Attorney Docket No. END9484USNP14/220491-14;
U.S. Patent Application, titled INTEGRAL CARTRIDGE STIFFENING FEATURES TO REDUCE CARTRIDGE DEFLECTION; Attorney Docket No. END9484USNP15/220491-15;
U.S. Patent Application, titled STAPLE CARTRIDGE COMPRISING WALL STRUCTURES TO REDUCE CARTRIDGE DEFLECTION; Attorney Docket No. END9484USNP16/220491-16;
U.S. Patent Application, titled PAN-LESS STAPLE CARTRIDGE ASSEMBLY COMPRISING RETENTION FEATURES FOR HOLDING STAPLE DRIVERS AND SLED; Attorney Docket No. END9484USNP17/220491-17;
U.S. Patent Application, titled STAPLE CARTRIDGE COMPRISING A SLED HAVING A DRIVER LIFT CAM; Attorney Docket No. END9484USNP18/220491-18;
U.S. Patent Application, titled SURGICAL STAPLE CARTRIDGES WITH SLEDS CONFIGURED TO BE COUPLED TO A FIRING DRIVER OF A COMPATIBLE SURGICAL STAPLER; Attorney Docket No. END9484USNP19/220491-19;
U.S. Patent Application, titled STAPLE CARTRIDGE COMPRISING A COMPOSITE SLED; Attorney Docket No. END9484USNP20/220491-20;
U.S. Patent Application, titled SURGICAL INSTRUMENTS WITH JAW AND FIRING ACTUATOR LOCKOUT ARRANGEMENTS LOCATED PROXIMAL TO A JAW PIVOT LOCATION; Attorney Docket No. END9484USNP21/220491-21;
U.S. Patent Application, titled SURGICAL INSTRUMENTS WITH LATERALLY ENGAGEABLE LOCKING ARRANGEMENTS FOR LOCKING A FIRING ACTUATOR; Attorney Docket No. END9484USNP22/220491-22;
U.S. Patent Application, titled DUAL INDEPENDENT KEYED LOCKING MEMBERS ACTING ON THE SAME DRIVE MEMBER; Attorney Docket No. END9484USNP23/220491-23;
U.S. Patent Application, titled ADJUNCTS FOR USE WITH SURGICAL STAPLING INSTRUMENTS; Attorney Docket No. END9484USNP24/220491-24;
U.S. Patent Application, titled ADJUNCTS FOR USE WITH SURGICAL STAPLING INSTRUMENTS; Attorney Docket No. END9484USNP25/220491-25;
U.S. Patent Application, titled ZONED ALGORITHM ADAPTIVE CHANGES BASED ON CORRELATION OF COOPERATIVE COMPRESSION CONTRIBUTIONS OF TISSUE; Attorney Docket No. END9484USNP27/220491-27;
U.S. Patent Application, titled STAPLE CARTRIDGES COMPRISING TRACE RETENTION FEATURES; Attorney Docket No. END9484USNP29/220491-29; and
U.S. Patent Application, titled STAPLE CARTRIDGES COMPRISING STAPLE RETENTION FEATURES; Attorney Docket No. END9484USNP30/220491-30.

Numerous specific details are set forth to provide a thorough understanding of the overall structure, function, manufacture, and use of the embodiments as described in the specification and illustrated in the accompanying drawings. Well-known operations, components, and elements have not been described in detail so as not to obscure the embodiments described in the specification. The reader will understand that the embodiments described and illustrated herein are non-limiting examples, and thus it can be appreciated that the specific structural and functional details disclosed herein may be representative and illustrative. Variations and changes thereto may be made without departing from the scope of the claims.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Various exemplary devices and methods are provided for performing laparoscopic and minimally invasive surgical procedures. However, the reader will readily appreciate that the various methods and devices disclosed herein can be used in numerous surgical procedures and applications including, for example, in connection with open surgical procedures. As the present Detailed Description proceeds, the reader will further appreciate that the various instruments disclosed herein can be inserted into a body in any way, such as through a natural orifice, through an incision or puncture hole formed in tissue, etc. The working portions or end effector portions of the instruments can be inserted directly into a patient's body or can be inserted through an access device that has a working channel through which the end effector and elongate shaft of a surgical instrument can be advanced.

A surgical stapling system can comprise a shaft and an end effector extending from the shaft. The end effector comprises a first jaw and a second jaw. The first jaw comprises a staple cartridge. The staple cartridge is insertable into and removable from the first jaw; however, a staple cartridge may not be removable from, or at least readily replaceable from, the first jaw. The second jaw comprises an anvil configured to deform staples ejected from the staple cartridge. The second jaw is pivotable relative to the first jaw about a closure axis; however, the first jaw may be pivotable relative to the second jaw. The surgical stapling system further comprises an articulation joint configured to permit the end effector to be rotated, or articulated, relative to the shaft. The end effector is rotatable about an articulation axis extending through the articulation joint. However, the surgical stapling system may not include an articulation joint.

The staple cartridge comprises a cartridge body. The cartridge body includes a proximal end, a distal end, and a deck extending between the proximal end and the distal end. In use, the staple cartridge is positioned on a first side of the tissue to be stapled and the anvil is positioned on a second side of the tissue. The anvil is moved toward the staple cartridge to compress and clamp the tissue against the deck. Thereafter, staples removably stored in the cartridge body can be deployed into the tissue. The cartridge body includes staple cavities defined therein wherein staples are removably stored in the staple cavities. The staple cavities are arranged in six longitudinal rows. Three rows of staple cavities are positioned on a first side of a longitudinal slot and three rows of staple cavities are positioned on a second side of the longitudinal slot. Other arrangements of staple cavities and staples may be possible.

The staples are supported by staple drivers in the cartridge body. The drivers are movable between a first, or unfired position, and a second, or fired, position to eject the staples from the staple cavities. The drivers are retained in the cartridge body by a retainer which extends around the bottom of the cartridge body and includes resilient members configured to grip the cartridge body and hold the retainer to the cartridge body. The drivers are movable between their unfired positions and their fired positions by a sled. The sled is movable between a proximal position adjacent the proximal end and a distal position adjacent the distal end. The sled comprises a plurality of ramped surfaces configured to slide under the drivers and lift the drivers, and the staples supported thereon, toward the anvil.

Further to the above, the sled is moved distally by a firing driver. The firing driver is configured to contact the sled and push the sled toward the distal end. The longitudinal slot defined in the cartridge body is configured to receive the firing driver. The anvil also includes a slot configured to receive the firing driver. The firing driver further comprises a first cam which engages the first jaw and a second cam which engages the second jaw. As the firing driver is advanced distally, the first cam and the second cam can control the distance, or tissue gap, between the deck of the staple cartridge and the anvil. The firing driver also comprises a knife configured to incise the tissue captured intermediate the staple cartridge and the anvil. It is desirable for the knife to be positioned at least partially proximal to the ramped surfaces such that the staples are ejected ahead of the knife.

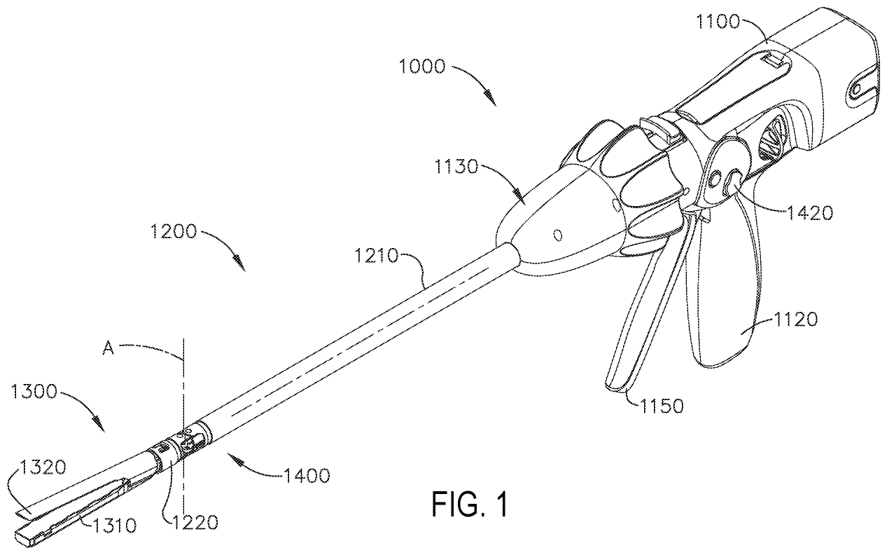

A surgical stapling instrument 1000 is illustrated in FIG. 1. The stapling instrument 1000 comprises a handle 1100, an elongate shaft 1200 extending from the handle 1100, and an end effector 1300 including a cartridge jaw 1310 and an anvil jaw 1320. The handle 1100 comprises a housing 1110 including a grip 1120 that is sized and configured to be held by a clinician. The handle 1100 further comprises triggers that are actuatable by the clinician to operate the stapling instrument 1000 as described further below. The stapling instrument 1000 can comprise a housing assembly configured to be attached to a robotic surgical instrument system instead of the handle 1100. As such, the housing assembly can comprise rotatable inputs that are operably coupled with motor-driven outputs of the robotic surgical instrument system when the housing assembly is attached to the robotic surgical instrument system. The robotic surgical instrument system comprises a control station including triggers that are actuatable by a clinician to operate the surgical instrument 1000. The entire disclosure of U.S. Pat. No. 9,072,535, entitled SURGICAL STAPLING INSTRUMENT WITH ROTATABLE DEPLOYABLE ARRANGEMENTS, which issued on Jul. 7, 2015, is incorporated by reference herein.

The elongate shaft 1200 is rotatable relative to handle 1100 about a longitudinal axis L. When the elongate shaft 1200 is rotated relative to the handle 1100, the end effector 1300 rotates with the elongate shaft 1200. The handle 1100 comprises a rotation actuator 1130 mounted to the elongate shaft 1200 that is rotatable relative to the handle housing 1110 by the clinician to rotate the shaft 1200 about the longitudinal axis L. The stapling instrument 1000 can comprise a motor-driven system that is operable to rotate the elongate shaft 1200. As such, the motor-driven system can comprise an electric motor mounted in the handle 1100 that includes an output gear meshingly engaged with a ring of gear teeth defined on the elongate shaft 1200. The motor-driven system further comprises a trigger, such as a switch, for example, accessible by the clinician operating the stapling instrument 1000. As such, the actuator can be positioned on the handle 1100.

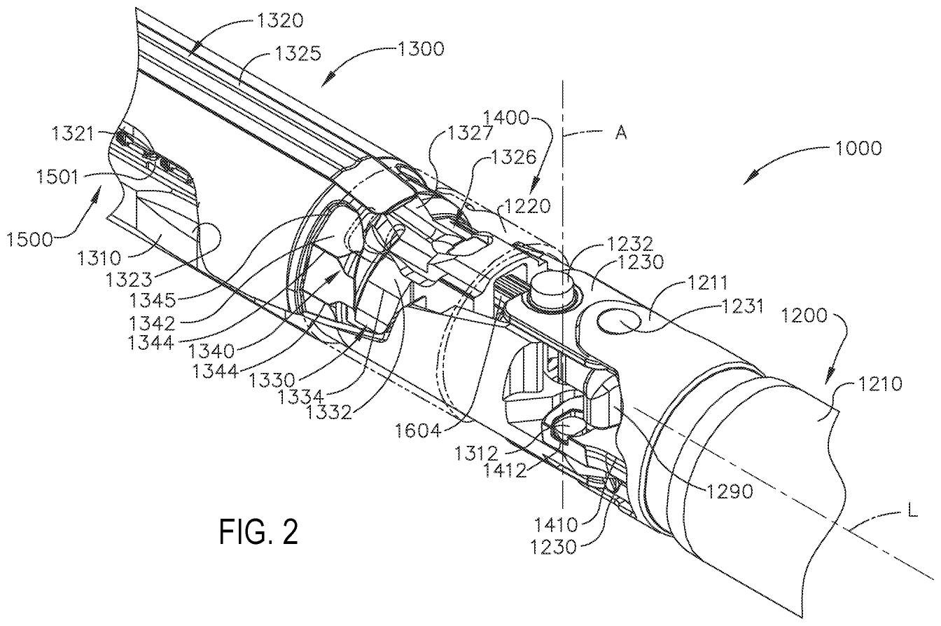

The end effector 1300 is rotatable relative to the shaft 1200 about an articulation joint 1400. The articulation joint 1400 defines an articulation axis A about which the end effector 1300 is articulated relative to the shaft 1200. The articulation joint 1400 also defines a plane within which the end effector 1300 is articulated relative to the shaft 1200. The shaft 1200 comprises a projection extending from a frame 1290 of the shaft 1200 that is closely received within an aperture defined in the cartridge jaw 1310 where the projection and the aperture define a pivot joint about which the end effector 1300 is articulated. The stapling instrument 1000 further comprises an articulation drive system. Referring to FIGS. 1 and 2, the articulation drive system comprises an articulation driver 1410, an electric motor operable to drive the articulation driver 1410 longitudinally, and a motor control circuit including a trigger 1420, such as a switch, for example, accessible by the clinician operating the stapling instrument 1000. The trigger 1420 is positioned on the handle 1100, but could be positioned in any suitable location on the stapling instrument 1000. The cartridge jaw 1310 comprises an articulation drive pin 1312 and the articulation driver 1410 comprises a distal end 1412 comprising an aperture defined therein within which the articulation drive pin 1312 is positioned. When the articulation driver 1410 is translated distally by the articulation drive system, the end effector 1300 rotates in a first direction and, when the articulation driver 1410 is translated proximally by the articulation drive system, the end effector 1300 rotates in a second, or opposite, direction. The articulation joint 1400 can define more than one articulation axis, such as two articulation axes, for example, about which the end effector 1300 can be rotated relative to the shaft 1200. The articulation joint 1400 can comprise a flexible articulation region. The entire disclosure of U.S. Pat. No. 9,629,629, entitled CONTROL SYSTEMS FOR SURGICAL INSTRUMENTS, which issued on Apr. 25, 2017, is incorporated by reference herein.

Further to the above, the end effector 1300 comprises a cartridge jaw 1310 and an anvil jaw 1320. Referring to FIG. 12, the cartridge jaw 1310 comprises a channel 1314 configured to receive a replaceable staple cartridge 1500. The channel 1314 comprises a bottom 1313 and lateral sidewalls 1315 extending upwardly from the bottom 1313. The staple cartridge 1500 comprises a cartridge body 1510 and a pan 1520 attached to the cartridge body 1510. That said, the staple cartridge 1500 may not comprise a pan. The cartridge body 1510 comprises a deck 1501 configured to support patient tissue and longitudinal rows of staple cavities defined in the deck 1501. The staple cartridge 1500 further comprises staples stored in the staple cavities and staple drivers movable within the staple cavities to eject the staples from the staple cavities during a staple firing stroke. The staple cartridge 1500 further comprises a sled 1530 (FIG. 12) movable from a proximal position to a distal position during the staple firing stoke to lift the staple drivers and eject the staples during the staple firing stroke. The staple cartridge 1500 and/or the cartridge jaw 1310 comprise features which releasably lock the staple cartridge 1500 in the cartridge jaw 1310 such that the staple cartridge 1500 is secured in position in the cartridge jaw 1310 but can be replaced during a surgical procedure. The staple cartridge 1500 can be secured in the cartridge jaw 1310 in a manner that does not allow the staple cartridge 1500 to be released from the cartridge jaw 1310 and replaced during a surgical procedure. The entire disclosure of U.S. Pat. No. 11,045,191, entitled METHOD FOR OPERATING A SURGICAL STAPLING SYSTEM, which issued on Jun. 29, 2021 is incorporated by reference herein.

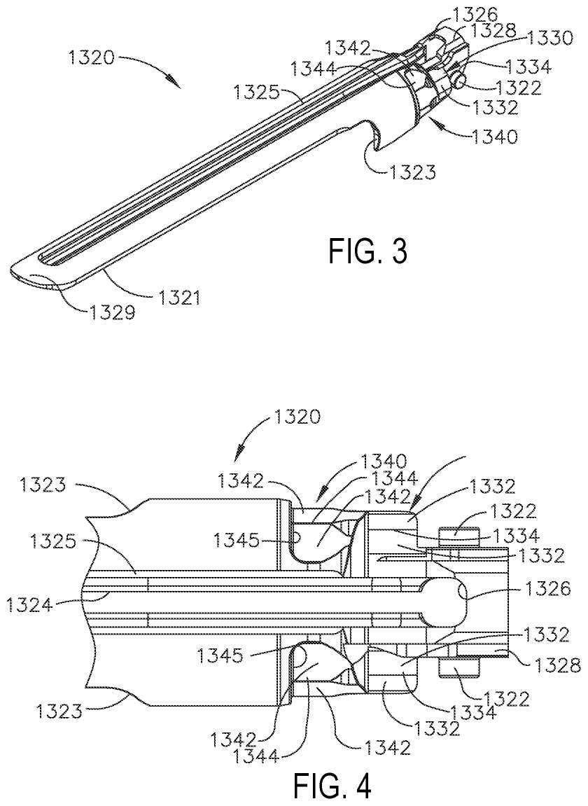

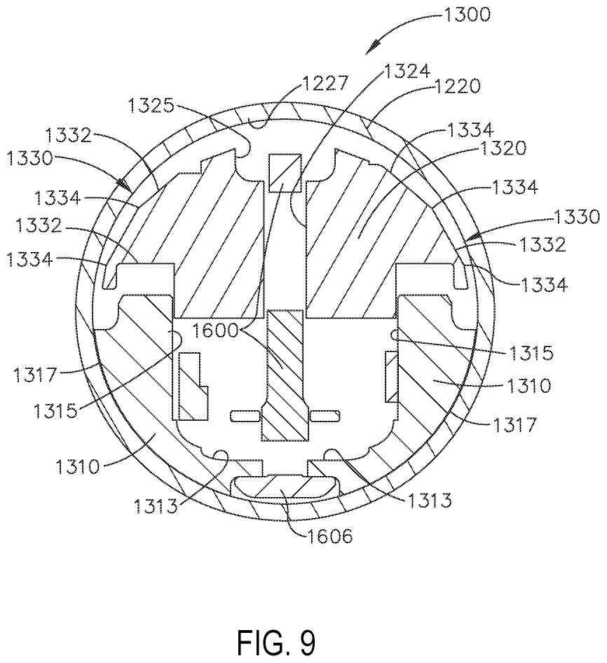

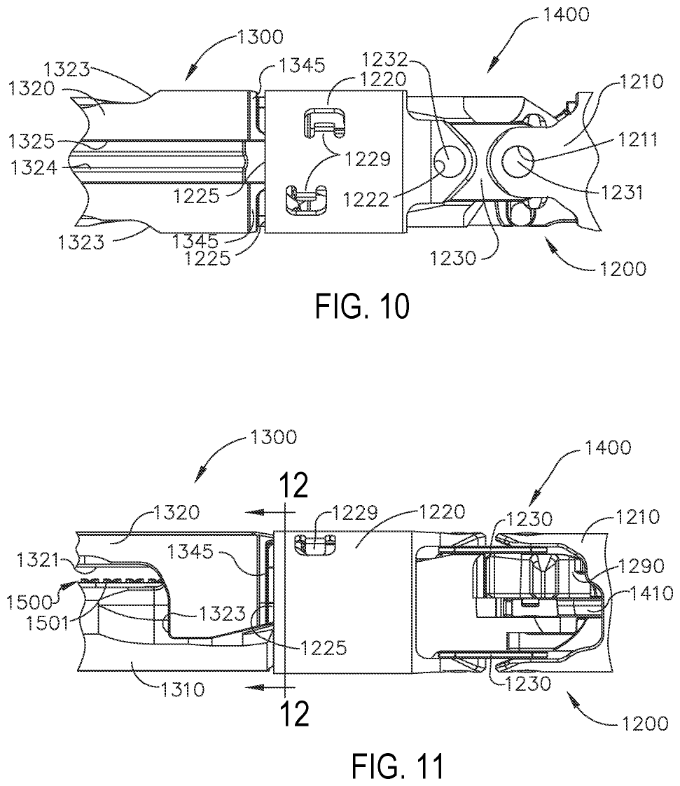

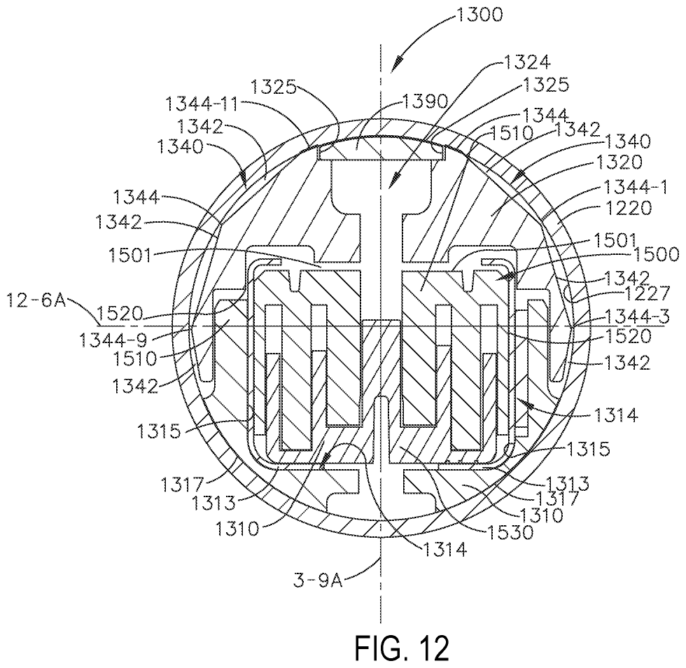

Further to the above, the anvil jaw 1320 is rotatably connected to the cartridge jaw 1310. Referring to FIGS. 3 and 4, the anvil jaw 1320 comprises pivot pins 1322 extending laterally therefrom. The pivot pins 1322 are rotatably secured in apertures defined in the cartridge jaw 1310 such that the anvil jaw 1320 rotates about a pivot axis PA. The anvil jaw 1320 further comprises a tissue compression surface 1321 that is brought into opposition with the deck 1501 of the staple cartridge 1500 when the anvil jaw 1320 is rotated between a fully open position (FIG. 8) and a fully closed position (FIG. 11). The anvil jaw 1320 further comprises staple forming pockets arranged in longitudinal rows that are registered with the staple cavities defined in the staple cartridge 1500 when the anvil jaw 1320 is in its fully-closed position. The anvil jaw 1320 further comprises tissue stops 1323 that extend downwardly toward the cartridge jaw 1310 and are positioned inwardly with respect to the lateral sides of the cartridge jaw 1310. The tissue stops 1323 of the anvil jaw 1320 can be positioned outwardly with respect to the lateral sides of the cartridge jaw 1310. The tissue stops 1323 inhibit the migration of tissue proximally into the end effector 1300 past the tissue stops 1323 which can reduce the possibility of the tissue coming into contact with a tissue cutting edge of a firing bar 1600 parked in a proximal unfired position (FIG. 2). The staple cartridge can comprise the tissue cutting edge; however, the tissue stops 1323 would still provide the same benefit. The firing bar 1600 comprises an elongate portion 1604 extending through the articulation joint 1400 and a cam that is at least partially positioned in a window 1326 defined through the top portion of the anvil jaw 1320 when the firing bar 1600 is in its proximal unfired position. When the firing bar 1600 is advanced distally during the staple firing stroke, the firing bar 1600 travels within a longitudinal slot 1324 defined in the top portion of the anvil jaw 1320. The anvil jaw 1320 further comprises a longitudinal recess 1325 that defines longitudinal shoulders on opposite sides of the longitudinal slot 1324 which are engaged by the cam to hold the anvil jaw 1320 in position during the staple firing stroke. Referring to FIG. 12, the anvil jaw 1320 further comprises a cap 1390 affixed thereto that extends over the longitudinal slot 1324 and the longitudinal recess 1325. Referring to FIG. 9, the firing bar 1600 further comprises a cam 1606 that engages the cartridge jaw 1310 during the staple firing stroke and co-operates with the anvil-engaging cam to hold the anvil jaw 1320 and the cartridge jaw 1310 in position relative to one another.

Further to the above, the anvil jaw 1320 is movable from a fully-open position (FIG. 8) to a fully-closed position (FIG. 11) to clamp tissue between the anvil jaw 1320 and the staple cartridge 1500. The stapling instrument 1000 further comprises a jaw closure system configured to move the anvil jaw 1320 into its fully-closed position. The jaw closure system comprises a trigger 1150 rotatably coupled to the handle 1100, a closure carriage positioned in the handle 1100 translatable distally by the trigger 1150, and a closure tube 1210 supported by the closure carriage such that the closure tube 1210 translates with the closure carriage. The trigger 1150 comprises a rotatable lever movable from an unactuated position to an actuated position to transmit a force applied to the trigger 1150 by a clinician to the closure tube 1210. When the trigger 1150 is pulled toward the grip 1120 by the clinician, the trigger 1150 pushes the closure carriage and the closure tube 1210 distally. The closure tube 1210 comprises a longitudinal aperture defined therein through which the shaft frame 1290 and the articulation driver 1410 extend. The jaw closure system further comprises a distal closure tube 1220 rotatably connected to the closure tube 1210 by two links 1230. Each link 1230 comprises a proximal projection 1231 closely received within an aperture 1211 defined in the closure tube 1210 and, in addition, a distal projection 1232 closely received within an aperture 1222 defined in the distal closure tube 1220. When the closure tube 1210 is driven distally by the jaw closure system, as described above, the closure tube 1210 pushes the links 1230 and the distal closure tube 1220 distally such that the closure tube 1210, the links 1230, and the distal closure tube 1220 translate relative to the articulation joint 1400. The links 1230 are sized and configured to permit the jaw closure drive to transmit a closing force across the articulation joint 1400 even when the end effector 1300 is in an articulated position.

The jaw closure system can comprise an electric motor configured to drive the closure tube 1210 distally through the closure stroke when the electric motor is operated in a first direction and retract the closure tube 1210 proximally when the electric motor is operated in a second, or opposite, direction. The jaw closure system can comprise a motor control circuit configured to control the operation of the electric motor. The motor control circuit comprises a processor and a trigger that is actuatable by the clinician to operate the electric motor.

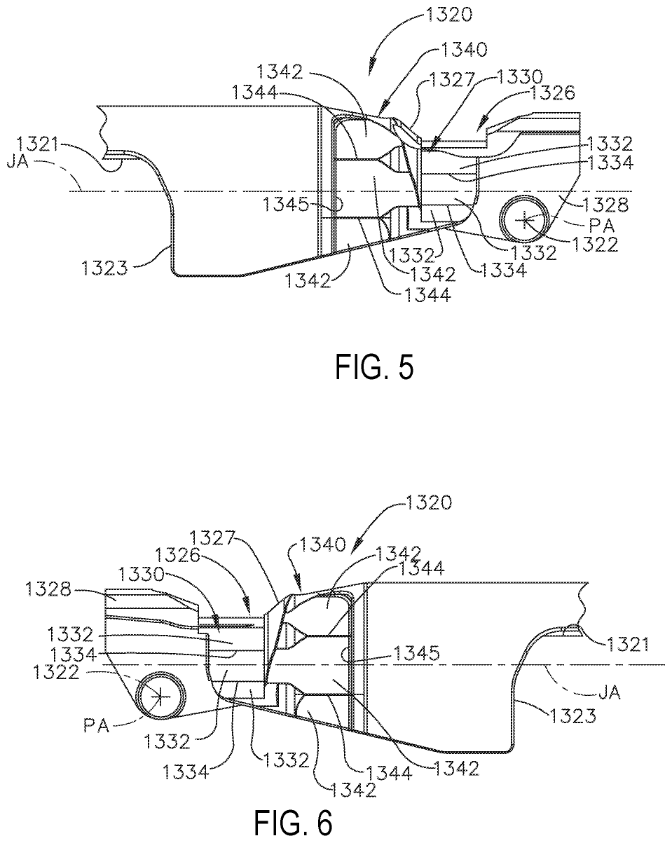

Referring to FIGS. 5 and 6, the anvil jaw 1320 comprises a proximal end 1328, a distal end 1329, and a jaw axis JA extending between a proximal end 1328 and a distal end 1329 of the anvil jaw 1320. The anvil jaw 1320 further comprises closure ramps 1327 defined thereon positioned distally with respect to the pivot pins 1322. When the distal closure tube 1220 is advanced distally by the closure drive during a closure stroke, further to the above, the distal closure tube 1220 contacts the closure ramps 1327 and moves the anvil jaw 1320 from its fully-open position into a partially-closed position. This partially-closed position comprises a state of closure intermediate the fully-open position and a fully-closed position. As the anvil jaw 1320 is advanced further distally during the closure stroke, the distal closure tube 1220 comes into contact with contact ridges 1344 defined on the anvil jaw 1320 to move the anvil jaw 1320 into a fully-closed position. More specifically, an inner surface 1227 of the distal closure tube 1220 contacts the contact ridges 1344 and slides along the contact ridges 1344 as the distal closure tube 1220 is moved to the end of the closure stroke. Referring primarily to FIG. 12, the inner surface 1227 of the distal closure tube 1220 is in contact with an outside surface 1317 of the cartridge jaw 1310 when the distal closure tube 1220 is in contact with the contact ridges 1344. The distal closure tube 1220 rotates the anvil jaw 1320 toward the cartridge jaw 1310 when the distal closure tube 1220 is moved distally during the closure stroke and holds the anvil jaw 1320 in position relative to the cartridge jaw 1310.

Referring primarily to FIGS. 4 and 12, further to the above, the contact ridges 1344 are arranged in an array extending about a periphery 1340 of the anvil jaw 1320. The top of the anvil jaw 1320, i.e., the portion of the anvil jaw 1320 facing away from the cartridge jaw 1310, has a generally arcuate shape defining an outer circumference and the contact ridges 1344 define part of the outer circumference. The contact ridges 1344 extend longitudinally and are parallel to one another and parallel to the jaw axis JA. The contact ridges 1344 can be substantially parallel to one another and are within 10 degrees of being parallel to the jaw axis JA. The periphery of the anvil jaw 1320 further comprises surfaces 1342 positioned intermediate the contact ridges 1344. The surfaces 1342 are flat; however, the surfaces 1342 can comprise any suitable shape, such as an arcuate shape, for example. Each surface 1342 extends between and connects adjacent contact ridges 1344. Referring primarily to FIG. 11, the distal closure tube 1220 is in contact with the contact ridges 1344, but not the surfaces 1342. Instead, clearance gaps are present between the surfaces 1342 and the inner surface 1227 of the distal closure tube 1220. As a result, the contact ridges 1344 comprise discrete control points between the distal closure tube 1220 and the anvil jaw 1320. A first set, or segment, of contact ridges 1344 are positioned on a first side of the jaw axis JA which comprise a first set of control points and a second set, or segment, of contact ridges 1344 are positioned on a second, or opposite, side of the jaw axis JA which comprise a second set of control points. Owing to the control points between the distal closure tube 1220 and the anvil jaw 1320, and the contact between the distal closure tube 1220 and the cartridge jaw 1310, the distal closure tube 1220 holds the anvil jaw 1320 in position relative to the cartridge jaw 1310. The first set of contact ridges 1344 and the second set of contact ridges 1344 are separated by a gap, or break, in the array of contact ridges 1344. Alternatively, the array of contact ridges 1344 may be continuous about the outer periphery of the anvil jaw 1320. The entire disclosure of U.S. Pat. No. 11,207,065, entitled METHOD FOR FABRICATING SURGICAL STAPLER ANVILS, which issued on Dec. 28, 2021 is incorporated by reference herein.

To insert the end effector 1300 into a patient, further to the above, the anvil jaw 1320 can be moved into a fully-closed position without patient tissue positioned between the anvil jaw 1320 and the staple cartridge 1500. For instance, the anvil jaw 1320 can be moved into a fully-closed so as to insert the end effector 1300 into a patient through a trocar, or cannula, for example. As such, the closure drive is configured to lock the anvil jaw 1320 in its fully-closed position. The handle 1100 can comprise a releasable lock configured to hold the closure trigger 1150 in a fully-actuated position to hold the anvil jaw 1320 in its fully-closed position. To open the anvil jaw 1320, the lock is releasable by the clinician which allows the closure trigger 1150 to return to its unactuated position via a return spring and/or the clinician moving the closure trigger 1150 back into its unactuated position. As the closure trigger 1150 is being returned back into its unactuated position, the closure trigger 1150 pulls the closure carriage, the closure tube 1210, the closure links 1230, and the distal closure tube 1220 proximally to disengage the distal closure tube 1220 from the contact ridges 1344 and the closure ramps 1327. The distal closure tube 1220 further comprises tabs 1229 extending radially inwardly toward the anvil jaw 1320. When the distal closure tube 1220 is being pulled proximally, the tabs 1229 are configured to contact the anvil jaw 1320 and pull, or positively open, the anvil jaw 1320. In addition to or in lieu of the positive jaw opening tabs 1229, the end effector 1300 can comprise one or more biasing members, such as springs, for example, configured to bias the anvil jaw 1320 into, or at least toward, its fully-open position.

Further to the above, the distal end 1329 of the anvil jaw 1320 is in contact with the staple cartridge 1500 when the anvil jaw 1320 is in its fully-closed position and the space between the anvil jaw 1320 and the staple cartridge 1500 is empty. As such, the tissue compression surface 1321 of the anvil jaw 1320 extends within a plane extending downwardly toward the staple cartridge 1500. The distal end 1329 of the anvil jaw 1320 may not be in contact with the staple cartridge 1500 when the anvil jaw 1320 is in its fully-closed position and the space between the anvil jaw 1320 and the staple cartridge 1500 is empty. However, patient tissue can be captured between the anvil jaw 1320 and the staple cartridge 1500 when the anvil jaw 1320 is moved into its fully-closed position. As such, the orientation of the anvil jaw 1320 relative to the staple cartridge 1500 depends on thickness and/or density of the tissue, for example, captured between the anvil jaw 1320 and the staple cartridge 1500. Thus, the anvil jaw 1320 may be in one orientation when the captured patient is thick and another orientation when the captured patient tissue is thin; however, both orientations, along with other orientations of the anvil jaw 1320, comprise fully-closed positions of the anvil jaw 1320.

At the end of the closure stroke, further to the above, a distal end 1225 of the distal closure tube 1220 is adjacent a ledge 1345 defined at the distal end of the contact ridges 1344. The distance between the distal end 1225 and the ledge 1345 at the end of the closure stroke depends on, among other things, the thickness and/or density of the patient tissue captured between the anvil jaw 1320 and the staple cartridge 1500. For instance, the distal end 1225 of the distal closure tube 1220 is positioned further away from the ledge 1345 when the captured patient tissue is thick as compared to when the captured patient tissue is thin. The distal end 1225 can come into contact with the ledge 1345 at the end of the closure stroke which can push the anvil jaw 1320 into its fully-closed position. The clinician can re-open the anvil jaw 1320 by reversing the operation of the electric motor via the control circuit actuator and, if desired, re-position the jaws 1310 and 1320 relative to the patient tissue.

When the anvil jaw 1320 is in a fully-closed position, further to the above, the staple firing system can be actuated to fire the staples from the staple cartridge 1500. The staple firing system comprises an electric motor and a control circuit configured to control the electric motor. The control circuit of the staple firing system comprises a processor and a trigger that, when actuated by a clinician, controls the operation of the electric motor to move the firing bar 1600 distally to perform a staple firing stroke. The firing bar 1600 comprises a distally-presented tissue cutting edge and, during the staple firing stroke, the patient tissue captured between the staple cartridge 1500 and the anvil jaw 1320 is stapled and incised. After the staple firing stroke has been performed, or at least partially performed, the electric motor can be operated in an opposite direction to retract the firing bar 1600. Once the firing bar 1600 has been sufficiently retracted, the anvil jaw 1320 can be re-opened to unclamp the stapled and incised patient tissue.

As described above, the surgical instrument 1000 is a stapling instrument. That said, the present disclosure can be adapted to any suitable surgical instrument. A suitable surgical instrument may comprise an electrocautery instrument that applies electrical energy to patient tissue, for example.

Further to the above, the anvil jaw 1320 comprises an array of ridges 1334. The ridges 1334 are positioned proximally with respect to the contact ridges 1344 and the array of ridges 1334 extends about a periphery 1330 of the anvil jaw 1320 that is proximal to the periphery including the contact ridges 1344. The periphery including the ridges 1334 has a generally arcuate shape defining an outer circumference and the ridges 1334 define part of the outer circumference. The ridges 1334 extend longitudinally and are parallel to one another and parallel to the jaw axis JA. The ridges 1334 can be substantially parallel to one another and are within 10 degrees of being parallel to the jaw axis JA. The periphery of the anvil jaw 1320 further comprises relief surfaces 1332 positioned intermediate the ridges 1334. The relief surfaces 1332 are flat; however, the relief surfaces 1332 can comprise any suitable shape, such as an arcuate shape, for example. Each relief surface 1332 extends between and connects adjacent ridges 1334.

Further to the above, the ridges 1334 are not in contact with the distal closure tube 1220 when the anvil jaw 1320 is in its fully-closed position or any of its partially-closed positions. Referring primarily to FIG. 9, however, the ridges 1334 can come into contact with the anvil jaw 1320 when the anvil jaw 1320 is in its fully-open position. That said, clearance gaps are present between the relief surfaces 1332 and the distal closure tube 1220 through out the entire range of motion of the anvil jaw 1320. A first set, or segment, of ridges 1334 are positioned on a first side of the jaw axis JA which comprise a first set of control points and a second set, or segment, of ridges 1334 are positioned on a second, or opposite, side of the jaw axis JA which comprise a second set of control points. Owing to the control points between the distal closure tube 1220 and the anvil jaw 1320 when the anvil jaw 1320 is in its fully-open position, the distal closure tube 1220 can limit the upward, or opening, rotation of the anvil jaw 1320. The first set of ridges 1334 and the second set of ridges 1334 are separated by a gap, or break, in the array of ridges 1334 but could comprise a continuous array without a gap, or break, therein. The above being said, the distal closure tube 1220 may engage the contact ridges 1344 to close the anvil jaw 1320 but does not engage the ridges 1334 of the anvil jaw 1320.

Referring to FIGS. 4-6, the closure ramps 1327 extend between and connect the array of ridges 1334 and relief surfaces 1332 and the array of contact ridges 1344 and surfaces 1342. That said, the closure ramps 1327 may be connected to the array of contact ridges 1344 and surfaces 1342 but not the array of ridges 1334 and relief surfaces 1332. Alternatively, the present disclosure may not comprise the closure ramps 1327. As such, a longitudinal gap may be present between the array of ridges 1334 and relief surfaces 1332 and the array of contact ridges 1344 and surfaces 1342.

Referring to FIGS. 4-6, the ridges 1334 are not aligned with the contact ridges 1344. The contact ridges 1344 extend about a first circumferential periphery of the anvil jaw 1320 and the ridges 1334 extend about a second circumferential periphery of the anvil jaw 1320 where the second circumferential periphery is closer to the longitudinal jaw axis JA than the first circumferential periphery. The ridges 1334 are closer to the longitudinal jaw axis JA than the contact ridges 1344 and, as a result, the ridges 1334 are longitudinally unaligned with the contact ridges 1344. The first circumferential periphery and the second circumferential periphery are centered about the longitudinal jaw axis JA; however, the first circumferential periphery and the second circumferential periphery can extend about, but may not be centered about, the longitudinal jaw axis JA. In addition, the ridges 1334 are declocked, or unregistered, with the contact ridges 1344 about the longitudinal jaw axis JA. When viewed distally end-on along the longitudinal jaw axis JA, the ridges 1334 are aligned with the surfaces 1342 and, similarly, the contact ridges 1344 are aligned with the relief surfaces 1332 and, as a result, the ridges 1334 are longitudinally unaligned with the contact ridges 1344. As a result of this arrangement, the relief surfaces 1332 create clearance gaps between the distal closure tube 1220 and the anvil jaw 1320 that are aligned with the contact ridges 1344. Such clearance gaps permit the distal closure tube 1220 to engage the contact ridges 1344 and control the anvil jaw 1320 without interference from the periphery including the ridges 1334.

As discussed above, the jaw closure system of the stapling instrument 1000 comprises, among other things, a closure tube 1210 and a distal closure tube 1220 rotatably connected to the closure tube 1210. As also discussed above, the distal closure tube 1220 engages the cartridge jaw 1310 and the anvil jaw 1320 during the closure stroke to position the anvil jaw 1320 relative to the cartridge jaw 1310. The distal closure tube 1220 defines a perimeter that entirely surrounds the cartridge jaw 1310 and the anvil jaw 1320; however, the distal closure tube 1220 may only partially surrounds the cartridge jaw 1310 and/or the anvil jaw 1320. As such, the distal closure tube 1220 can comprise one or more apertures defined therein. The distal closure tube 1220 can comprise a first cantilever that engages the cartridge jaw 1310 and a second cantilever that engages the anvil jaw 1320 where the distal ends of the first cantilever and the second cantilever are unconnected. The jaw closure drive can comprise any suitable closure driver that engages the anvil jaw 1320 and moves the anvil jaw 1320 into a closed position. The closure driver can comprise a closure bar that engages an outer perimeter of the cartridge jaw 1310 and/or anvil jaw 1320. One or both of the ridges 1334 and 1344 may not be defined on the outer perimeter of the anvil jaw 1320 and are instead defined in an internal cavity defined in the anvil jaw 1320. As such, the closure driver, such as a bar, for example, enters into the internal cavity defined in the anvil jaw 1320 to engage the ridges 1334 and/or 1344, and/or any other suitable contact surfaces.

As described above, the anvil jaw 1320 is rotatable relative to the cartridge jaw 1310. Alternatively, the cartridge jaw 1310 can be rotatable relative to the anvil jaw 1320. As such, the cartridge jaw 1310 can comprise an array of ridges 1334 and relief surfaces 1332 and an array of contact ridges 1344 and surfaces 1342. Similar to the above, the distal closure tube 1320 is configured to engage the contact ridges 1344 on the cartridge jaw 1310 and move the cartridge jaw 1310 into a closed position opposite the anvil jaw 1320. The cartridge jaw 1310 and the anvil jaw 1320 can both be movable relative to one another. As such, the cartridge jaw 1310 can comprise an array of ridges 1334 and relief surfaces 1332 and an array of contact ridges 1344 and surfaces 1342 and, also, the anvil jaw 1320 can comprise an array of ridges 1334 and relief surfaces 1332 and an array of contact ridges 1344 and surfaces 1342. Similar to the above, the distal closure tube 1320 is configured to engage the contact ridges 1344 on the cartridge jaw 1310 and the anvil jaw 1320 to move the cartridge jaw 1310 and the anvil jaw 1320 into closed positions.

Referring again to FIGS. 4-6, adjacent surfaces 1342 extend at an angle to one another. Each contact ridge 1344 is defined as an edge extending between adjacent surfaces 1342. The surfaces 1342 can be ground flat and the ridges 1344 comprise sharp edges defined between the surfaces 1342. As such, a discrete line, or discrete lines, is provided in which the distal closure tube 1220 contacts. The contact ridges 1344 can comprise radiused, or rounded, edges extending between adjacent surfaces 1342. As such galling and/or sticking between the distal closure tube 1220 and the anvil jaw 1320 can be reduced. Similarly, adjacent relief surfaces 1332 extend at an angle to one another. Each contact ridge 1334 is defined as an edge extending between adjacent relief surfaces 1332. The relief surfaces 1332 can be ground flat and the ridges 1334 can comprise sharp edges defined between the relief surfaces 1332. The ridges 1334 can also comprise radiused, or rounded, edges extending between adjacent relief surfaces 1332.

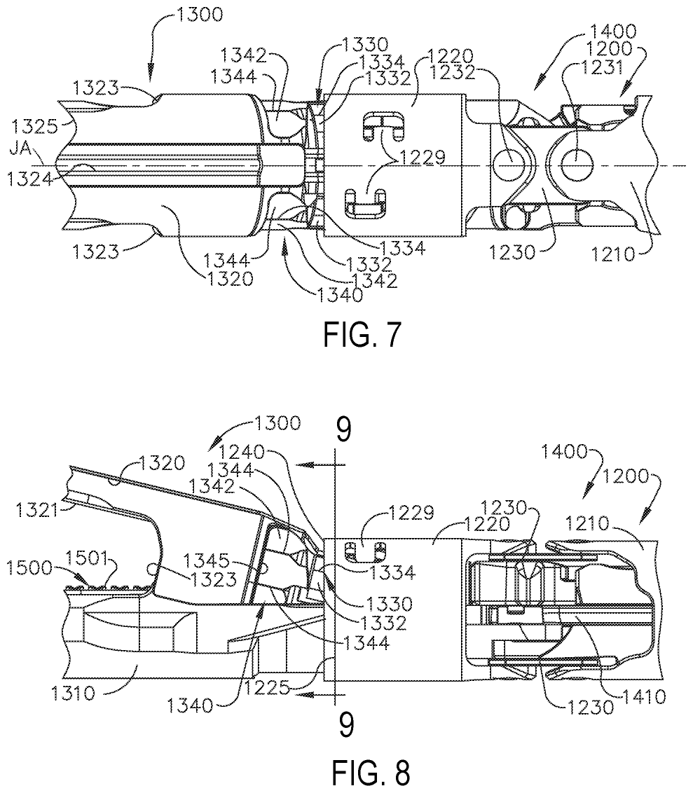

Further to the above, the relief surfaces 1332 allow the anvil jaw 1320 to move between its fully-open and fully-closed positions without the anvil jaw 1320 getting jammed during its open and closing motions. As discussed above, referring again to FIG. 7, the jaw tabs 1229 of the distal closure tube 1220 engage the anvil jaw 1320 as the distal closure tube 1220 is pulled proximally to rotate the anvil jaw 1320 into its fully-open position. As illustrated in FIG. 7, one of the jaw opening tabs 1229 is positioned distally with respect to the other. As also illustrated in FIG. 7, the distal jaw opening tab 1229 is positioned on a first lateral side of the distal closure tube 1220 and the proximal jaw opening tab 1229 is positioned on a second lateral side of the distal closure tube 1220. Stated another way, the jaw opening tabs 1229 are staggered longitudinally and off-center with respect to the jaw axis JA. Owing to this arrangement, the jaw opening tabs 1229 contact the anvil jaw 1320 sequentially as the distal closure tube 1220 is retracted. As the distal closure tube 1220 is retracted during an initial jaw opening motion, the distal jaw tab 1229 contacts the anvil jaw 1320 without the proximal jaw tab 1229 being in contact with the anvil jaw 1320. As such, the distal jaw tab 1229 pulls the anvil jaw 1320 into a partially open position by itself. As the distal closure tube 1220 is retracted further, the proximal jaw tab 1229 comes into contact with the anvil jaw 1320 and pulls the anvil jaw 1320 into its fully-open position during a second opening motion. The initial jaw opening motion and the second opening motion may comprise one continuous motion, while a pause may be present between the initial jaw opening motion and the second opening motion. The distal jaw tab 1229 disengages from the anvil jaw 1320 before or when the proximal jaw tab 1229 comes into contact with the anvil jaw 1320. The distal jaw tab 1229 and the proximal jaw tab 1229 can be simultaneously in contact with the anvil jaw 1320 for at least a portion of the second opening motion. In either event, the sequential co-operation of the two jaw opening tabs 1229 allows the anvil jaw 1320 to be opened wider than could be done with a single jaw opening tab. A wider open position of the anvil jaw 1320 creates a larger space, or jaw aperture, between the anvil jaw 1320 and the cartridge jaw 1310 that facilitates the positioning of the patient tissue between the jaws 1310 and 1320. Further to the above, the longitudinally staggered and laterally offset arrangement of the jaw opening tabs 1229 allow for this advantage to be obtained; however, other arrangements could be used. Concurrent with the wider fully-open position of the anvil jaw 1320 provided by the arrangement of the jaw opening tabs 1229, the relief surfaces 1332 of the anvil jaw 1320 provide sufficient clearance for the anvil jaw 1320 to move into such a wider fully-open position. The entire disclosures of U.S. Pat. No. 10,182,818, entitled SURGICAL END EFFECTORS WITH POSITIVE JAW OPENING ARRANGEMENTS, which issued on Jan. 22, 2019, U.S. Pat. No. 10,758,232, entitled SURGICAL INSTRUMENT WITH POSITIVE JAW OPENING FEATURES, which issued on Sep. 1, 2020, U.S. Pat. No. 11,207,065, entitled METHOD FOR FABRICATING SURGICAL STAPLER ANVILS, which issued on Dec. 28, 2021, and U.S. Patent Application Publication No. 2018/0168575, entitled SURGICAL STAPLING SYSTEMS, which published on Jun. 21, 2018, are incorporated by reference herein.

Further to the above, the proximal jaw tab 1229 can contact the anvil jaw 1320 without the distal jaw tab 1229 being in contact with the anvil jaw 1320 when the closure tube 1220 is initially retracted. As such, the proximal jaw tab 1229 pulls the anvil jaw 1320 into a partially open position by itself. As the distal closure tube 1220 is retracted further, the distal jaw tab 1229 comes into contact with the anvil jaw 1320 and pulls the anvil jaw 1320 into its fully-open position during a second opening motion. The initial jaw opening motion and the second opening motion may comprise one continuous motion, while a pause may be present between the initial jaw opening motion and the second opening motion. The proximal jaw tab 1229 disengages from the anvil jaw 1320 before or when the distal jaw tab 1229 comes into contact with the anvil jaw 1320. The distal jaw tab 1229 and the proximal jaw tab 1229 can be simultaneously in contact with the anvil jaw 1320 for at least a portion of the second opening motion. In either event, the sequential co-operation of the two jaw opening tabs 1229 allows the anvil jaw 1320 to be opened wider than could be done with a single jaw opening tab.

Further to the above, referring again to FIG. 12, the anvil jaw 1320 is movable between open and closed positions along an axis 12-6A which represents, in FIG. 12, a line extending between the 12 o'clock and 6 o'clock positions on a clock face. As discussed above, the distal closure tube 1220 is advanced distally into contact with the anvil jaw 1320 to move the anvil jaw 1320 into a closed position. More specifically, as also discussed above, the distal closure tube 1220 is advanced distally into contact with ridges 1344 and then slides along the ridges 1344 to move and hold the anvil jaw 1320 in its fully-closed position. When patient tissue is compressed between the anvil jaw 1320 and the staple cartridge 1500, however, the anvil jaw 1320 is biased away from the staple cartridge 1500 upwardly along the 12-6A axis by the compressed patient tissue. Similarly, the staples forming against the anvil jaw 1320 during the staple firing stroke tend to push the anvil jaw 1320 away from the staple cartridge 1500. As such, absent more, the ridges demarcated 1344-1 (representing the 1 o'clock position of a clock face) and 1344-11 (representing the 11 o'clock position of a clock face) in FIG. 12 apply a large opening force to the distal closure tube 1210 that tends to pull the distal closure tube 1220 against the cartridge jaw 1310 and stretch, elastically and/or plastically, the distal closure tube 1220 into an ovaloid shape. More specifically, the distal closure tube 1220, which has a round shape in its unloaded state, elongates along the 12-6A axis but contracts along an orthogonal 3-9A axis. Such stretching of the distal closure tube 1220 can allow the anvil jaw 1320 to at least partially open and may increase the force needed to advance the firing driver through the staple firing stroke.

In order to reduce, if not eliminate, the ovaloid stretching of the distal closure tube 1220, further to the above, the anvil jaw 1320 comprises ridges 1344-3 and 1344-9 which maintain, or at least substantially maintain, the round configuration of the distal closure tube 1220 seen in FIG. 12. The ridge 1344-3 represents the 3 o'clock position of a clock face in FIG. 12 and the ridge 1344-9 represents the 9 o'clock position of a clock face in FIG. 12. The ridges 1344-3 and 1344-9 comprise the outermost lateral edges of the anvil jaw 1320 and define the 3-9A axis, mentioned above, which is orthogonal to the 12-6A axis. When, or if, the distal closure tube 1220 is stretched along the 12-6A axis by the anvil jaw 1320, as discussed above, the lateral sides of the distal closure tube 1220 come into contact with and are supported by the lateral ridges 1344-3 and 1344-9 which prevent, or at least limit, the inward lateral deflection of the lateral sides of the distal closure tube 1220. As such, the circular shape of the distal closure tube 1220 seen in FIG. 12 is maintained, or at least substantially maintained, by the lateral ridges 1344-3 and 1344-9 of the anvil jaw 1320, the anvil jaw 1320 is held in its fully-closed position, and, as a result, the force needed to fire the staple cartridge 1500 is reduced.

Further to the above, there is a line-to-line fit between an outer diameter of the anvil jaw 1320 defined between the lateral ridges 1344-3 and 1344-9 and an inner aperture diameter defined by the inner surface 1227 of the distal closure tube 1220. As such, the lateral ridges 1344-3 and 1344-9 oppose the inward lateral deflection of the lateral sides of the distal closure tube 1220 through out the entire closure stroke of the distal closure tube 1220. As the reader will appreciate, however, manufacturing tolerances may, absent more, result in the outer diameter of the anvil jaw 1320 defined between the lateral ridges 1344-3 and 1344-9 being smaller than the inner aperture diameter of the distal closure tube 1220. As such, the lateral sides of the distal closure tube 1220 will deflect into contact with the lateral ridges 1344-3 and 1344-9 which will then oppose the continued inward deflection of the lateral sides of the distal closure tube 1220. Such instances can be acceptable; however, they can be avoided by designing the anvil jaw 1320 and the distal closure tube 1220 such that there is contact between the lateral ridges 1344-3 and 1344-9 and the distal closure tube 1220 through out the tolerance ranges of the components. Such instances can also be avoided by establishing the outer diameter defined by the lateral ridges 1344-3 and 1344-9 as a jaw reference datum used to manufacture the anvil jaw 1320 and the inner diameter of the distal closure tube 1220 as a tube reference datum used to manufacture the distal closure tube 1220. Establishing these features as reference datums during the manufacturing process can assure that the line-to-line fit therebetween is maintained.

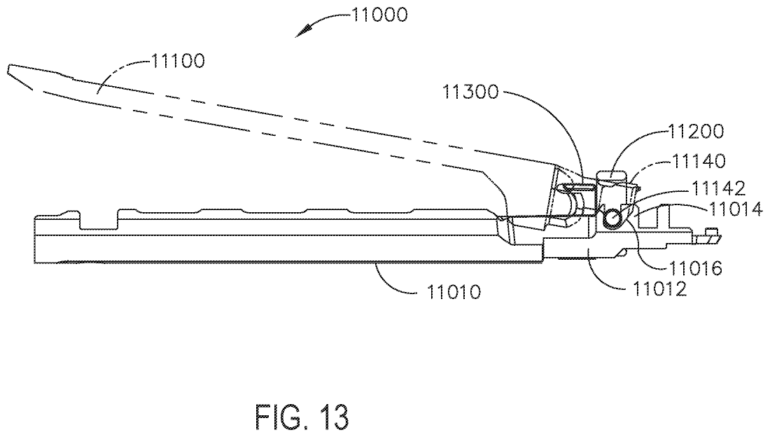

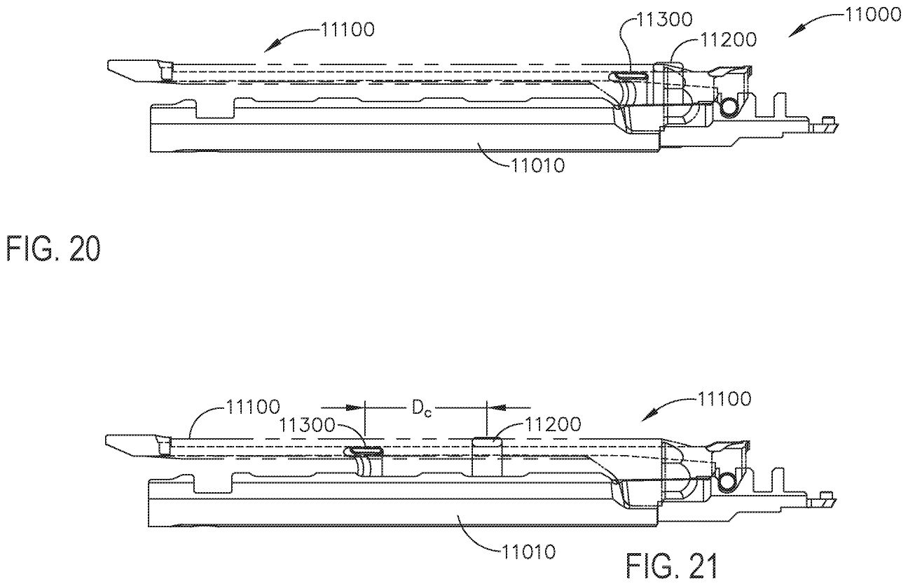

Referring to FIGS. 13-21, a surgical end effector 11000 comprises an anvil 11100 that is pivotally supported on an elongate channel 11010 that is configured to operably support a surgical staple cartridge therein. This arrangement also employs two rotary actuation shafts-one for closure, i.e., moving the anvil 11100 into a closed position, and one for firing, i.e., axially moving a firing member within the anvil 11100 and elongate channel 11010. The anvil 11100 includes an elongate anvil body 11110 and an anvil mounting portion 11140. For example, the anvil 11100 can be fabricated using various fabricating techniques described in U.S. patent application Ser. No. 16/105,101, entitled METHOD FOR FABRICATING SURGICAL STAPLER ANVILS. The entire disclosure of U.S. patent application Ser. No. 16/105,101, entitled METHOD FOR FABRICATING SURGICAL STAPLER ANVILS is hereby incorporated by reference herein. A pair of anvil trunnions 11142 protrude laterally from the anvil mounting portion 11140. Each anvil trunnion 11142 is pivotally supported in a corresponding trunnion cradle 11016 that is formed in a corresponding upstanding wall 11014 of a proximal end portion 11012 of the elongate channel 11010. See FIG. 13. The proximal end portion 11012 of the elongate channel 11010 is pivotally coupled to an elongate shaft assembly of a surgical instrument to facilitate the articulation of the end effector 11000. In other arrangements, the elongate channel 11010 is not capable of articulation. Each anvil trunnion 11142 is retained within its trunnion cradle 11016 by an anvil retainer (not shown).

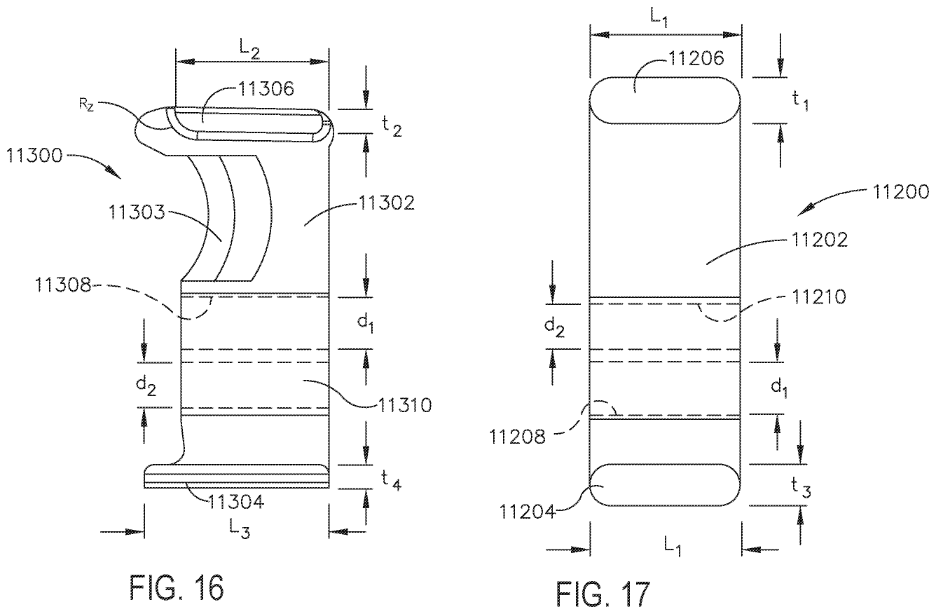

The surgical end effector 11000 includes an anvil closure member 11200 and a firing member 11300 that are each independently controlled and axially movable. FIG. 16 illustrates one form of a closure member 11200 that may be employed. As can be seen in FIG. 16, the closure member 11200 includes a vertically extending closure body 11202 that has two bottom channel tabs 11204 laterally protruding therefrom. A portion of the closure body 11202 is configured to extend through a slot in a bottom surface of the elongate channel 11010 and the channel tabs 11204 extend laterally outward to slideably engage the bottom of the channel 11010. Similarly, a pair of anvil engaging tabs 11206 protrude laterally from the top of the closure body 11202 to slideably engage the anvil 11100. The closure body 11202 includes a threaded through hole 11208 for threadably engaging a threaded portion of a rotatable closure drive shaft for axially driving the closure member 11200.

As indicated above, the surgical end effector 11000 further includes an axially movable firing member 11300. FIG. 17 illustrates one form of a firing member 11300 that may be employed. As can be seen in FIG. 17, the firing member 11300 includes a vertically extending firing member body 11302 that has a tissue cutting surface 11303 as well as two bottom channel tabs 11304 laterally protruding from the firing member body 11302. A portion of the firing member body 11302 is configured to extend through the slot 11019 in the bottom surface 11018 of the elongate channel 11010 and the channel tabs 11304 extend laterally outward to slideably engage the bottom of the channel 11010. See FIG. 17. Similarly, a pair of anvil engaging tabs 11306 protrude laterally from the top of the firing member body 11302 to slideably engage the anvil 11100.

Further to the above, the firing member body 11302 includes a threaded through hole 11308 that is threadably engaged with a threaded portion of a rotatable firing drive shaft. The firing drive shaft passes through an unthreaded clearance hole 11210 in the closure body 11202. See FIG. 17. The firing drive shaft extends axially down the elongate channel 11010 and is rotatably supported at a distal end portion thereof by a bearing (not shown) or another arrangement. Similarly, the closure drive shaft extends axially down the elongate channel 11010 and is rotatably supported at a distal end portion thereof by a bearing (not shown) or another arrangement. Thus, the firing member body 11302 similarly has an unthreaded clearance hole 11310 therethrough to accommodate the closure drive shaft. It will be appreciated that, in such arrangements, the closure drive shaft 10710 and the firing drive shaft 10610 may be supported in a vertical stacked arrangement so that they may be independently rotatable.

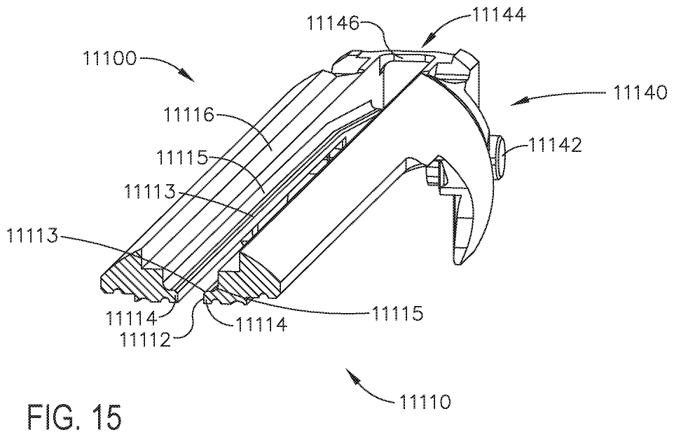

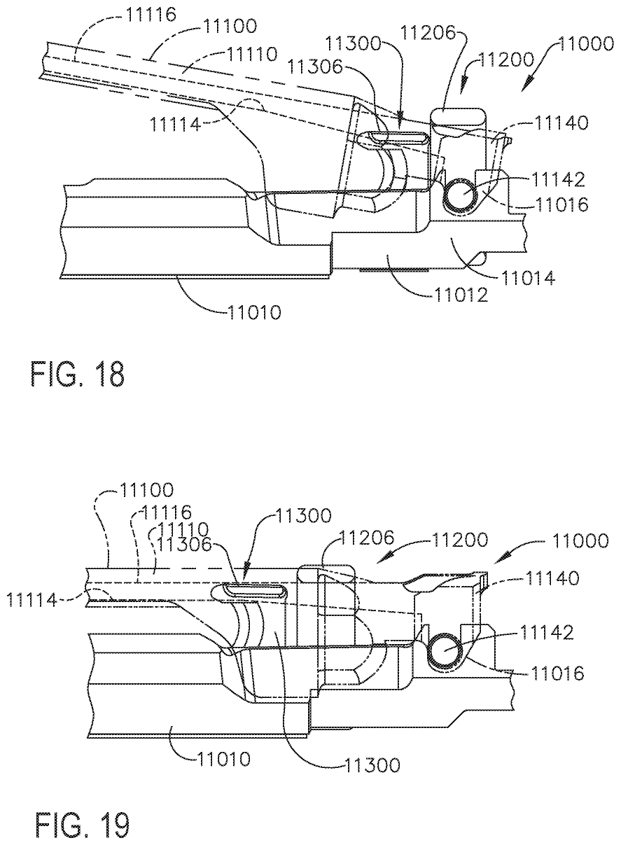

Referring to FIG. 18, the anvil mounting portion 11140 of the anvil 11100 includes a central cross brace 11144 that serves to define an opening 11146 for accommodating the closure member 11200 therein when the closure member 11200 is in its proximal-most position which corresponds to an open position of the anvil 11100. As can be seen in FIG. 18, the anvil body 11110 defines an elongate slot 11112 for accommodating the firing member body 11302 and closure body 11202 therethrough. The firing member 11300 is located distal to the closure member 11200. The anvil engagement tabs 11306 on the firing member 11302 are configured to slideably engage corresponding first or lower ledges 11114 that are formed on each side of the slot 11112. As can be seen in FIGS. 18 and 19, the lower ledges 11114 taper slightly downward at their proximal ends to accommodate the anvil engagement tabs 11306 on the firing member 11300 when the anvil is pivoted to its open position. The anvil engagement tabs 11206 on the closure member 11200 are configured to slideably engage corresponding second or higher ledges 11116 that are formed on each side of the slot 11112. See FIG. 15. To open the anvil 11100, the closure drive shaft is rotated to threadably drive the closure member 11200 proximally into its proximal-most position (FIG. 18). When the closure member 11200 is in its proximal-most position, the anvil engagement tabs 11206 thereon apply a pivotal opening motion to the anvil 11100 to pivot the anvil open. The firing member 11300 is in its starting position so that the anvil engagement tabs 11306 of the firing member 11300 do not apply a closure motion to the anvil 11100.

To close the anvil 11100, the closure drive shaft is rotated in an opposite direction to distally advance the closure member 11200 into a closed position. The firing drive shaft may also be simultaneously rotated to distally advance the firing member 11300 into a starting position. When the closure member 11200 and the firing member 11300 are in those positions, the anvil 11100 is closed and the firing member 11300 is ready to be fired. Thus, assuming that an unspent surgical staple cartridge has been first operably supported in the elongate channel 11010 and the end effector 11000 was manipulated to capture the target tissue between the staple cartridge and the anvil, the user may close the anvil 11100 onto the tissue in the above described manner to ready the end effector to be fired. During this closing process, the firing drive shaft is rotated to drive the firing member 11300 distally into the clamped tissue to cut the tissue and cause the staples stored in the staple cartridge to be formed into the cut tissue on both sides of the cut. During this process, the closure member 11200 may also be driven distally to apply additional closure motions to the anvil 11100 and elongate channel 11010. Depending upon the amount of resistance experienced by the firing member 11300, for example, the closure member 11200 can be advanced with the firing member 11300, stop and then go again. The closure member 11200 may be advanced distally at a different rate from the firing member's rate of distal advancement. The distance Dc between the closure member 11200 and the firing member 11300 may be controlled to balance the loads experienced during the firing process. See FIG. 21. For example, if the user wanted to decrease an amount of vertical load being experienced by the firing member 11300, the closure member 11200 could be moved closer to the firing member 11300 during advancement. The vertical loads experienced by the firing member 11300 may be increased by increasing the distance between the firing member 11300 and the closure member 11200.

Returning to FIGS. 16 and 17, the thickness t₁ of the anvil engagement tabs 11206 on the closure member 11200 is greater than the thickness t₂ of the anvil engagement tabs 11306 on the firing member 11300. The length L₁ of the anvil engagement tabs 11206 on the closure member 11200 may be slightly less than the length L₂ of the anvil engagement tabs 11306 on the firing member 11300. Likewise, the thickness t₃ of the channel tabs 11204 on the closure member 11200 may be greater than the thickness t₄ of the channel tabs 11304 on the firing member 11300. The length L₁ of the channel tabs 11204 on the closure member 11200 may be shorter than the length L₃ of the channel tabs 11304 on the firing member 11300. In both cases, the diameters d₁ of the threaded holes 11208, 11308 may be greater than the diameters d₂ of the unthreaded through holes 11210, 11310. In addition, the relative attack angles between the anvil engagement tabs 11206, 11306 and their corresponding anvil ledges and the channel tabs 11204, 11304 and their corresponding channel ledges may be varied, the same or different. The anvil engagement tabs 11306 on the firing member 11300 can be arranged at a slightly higher attack angle relative to their corresponding anvil ledges than the attack angle of the anvil engagement tabs 11206 on the closure member 11200. The channel tabs 11204 and 11304 can ride on the same ledges that are formed in the bottom of the elongate channel 11010. See FIG. 13. The closure member 11200 and the firing member 11300 have separate acting paths which can permit the closure member to be designed to accommodate larger moment arms from the anvil pivot for better firing efficiency.

One advantage that may be experienced when using the foregoing configuration is that the closure member 11200 can be moved away from the firing member 11300 to gain a significant amount of mechanical advantage during closure. The closure member 11200 does not need to travel the complete length of the firing stroke. For example, if the closure member 11200 were to be advanced about half way down the end effector, the relative stiffness of the anvil 11100 would reduce the amount of load being encountered by the firing member 11300. A control system employing sensors (e.g., strain gauges, etc.) for detecting amounts of loads being experienced by the firing system components and closure system components, as well as algorithms, can be used to balance the loads being encountered by both systems. For example, a maximum threshold of vertical load experienced by the firing member 11300 can be set in the controller based on the geometry and composition of that firing member component. When the load approaches that threshold, the algorithm can automatically advance the closure member 11200 so that it absorbs more of the load and reduces the amount of load being experienced by the firing member 11300. As the firing member 11300 is distally driven through the surgical staple cartridge, the firing member 11300, through the engagement of the anvil engagement tabs 11306 with the anvil 11100 and the engagement of the channel engagement tabs 11304 with the channel 11010, may serve to maintain a desired amount of tissue gap between a deck surface on the staple cartridge and a staple forming undersurface on the anvil 11100. Other closure control methods may also be employed in connection with opening and closing the end effector such as those disclosed in U.S. patent application Ser. No. 16/105,081, entitled METHOD FOR OPERATING A POWERED ARTICULATABLE SURGICAL INSTRUMENT. The entire disclosure of U.S. patent application Ser. No. 16/105,081, entitled METHOD FOR OPERATING A POWERED ARTICULATABLE SURGICAL INSTRUMENT is hereby incorporated by reference herein. The entire disclosure of U.S. Pat. No. 11,589,865, entitled METHODS FOR CONTROLLING A POWERED SURGICAL STAPLER THAT HAS SEPARATE ROTARY CLOSURE AND FIRING SYSTEMS, which issued on Feb. 28, 2023, is incorporated by reference herein. The entire disclosure of U.S. Pat. No. 6,978,921, entitled SURGICAL STAPLING INSTRUMENT INCORPORATING AN E-BEAM FIRING MECHANISM, which issued on Dec. 27, 2005, is incorporated by reference herein.

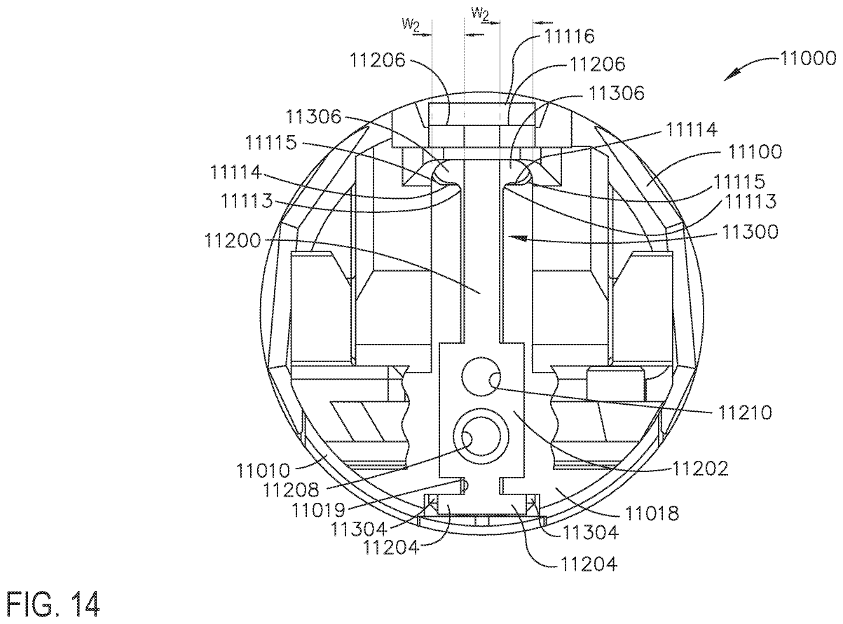

As discussed above, referring again to FIGS. 14, 15 and 17, the firing member 11300 engages the lower ledges 11114 of the anvil 11100 during a staple firing stroke to hold the anvil 11100 in position relative to the cartridge jaw 11010. As also discussed above, the firing member 11300 has two laterally-extending engagement tabs 11306-one that extends in a first lateral direction to engage a first lower ledge 11114 and another that extends in a second, or opposite, lateral direction to engage a second lower ledge 11114. Each engagement tab 11306 extends from the firing member body 11302 a width w₂. Notably, the width w₂ of each engagement tab 11306 is shorter than the length L₁. The width w₂ of each engagement tab 11306 can be between 15% and 25% of length L₁, between 20% and 30% of the length L₁, or between 25% and 35% of the length L₁, for example. As a result, each engagement tab 11306 has the configuration of a stubby flange that resists being deflected during the staple firing stroke. More specifically, the staples, when fired, are driven against the anvil 11100 during the staple firing stroke which tends to push the anvil 11100 away from the cartridge jaw 11010 and deflect the engagement tabs 11306 upwardly, i.e., away from the cartridge jaw 11010. Given the stubby configuration of the engagement tabs 11306, however, the engagement tabs 11306 resist the upward deflection thereof and, as a result, resist the upward movement of the anvil 11100 away from its fully-closed position during the staple firing stroke. As a result, the anvil 11100 can remain in its fully-closed position during the staple firing stroke and the staple firing drive does not need to transmit an excessive firing force into the firing member 11300 to push the anvil 11100 back into its fully-closed position as the firing member 11300 is moved distally.

As discussed above, referring again to FIGS. 14, 15 and 17, each engagement tab 11306 extends laterally from the firing member body 11302. A fillet radius R₂ is present at the interconnection between each engagement tab 11306 and the firing member body 11302 which resists the upward deflection of the engagement tab 11306 during the staple firing stroke. The fillet radius R₂ can be between 1/4 and 1/3 of the engagement tab thickness t₂, between 1/5 and 1/3 of the engagement tab thickness t₂, or between 1/5 and 1/3 of the engagement tab thickness t₂, for example. The fillet radius R₂ is present at a location with a high amount of strain and stress when the engagement tab 11306 is being bent upwardly. The fillet radius R₂ relieves part of that strain. Moreover, the anvil 11100 further comprises a radiused edge 11113 extending along each lower ledge 11114 that matches, or at least substantially matches, the fillet radius R₂ of the firing member 11300. The matched radiused edge 11113 may be defined by the fillet radius R₂. Matched radiused edge 11113 may be defined by a radius that is within 10% larger or within 10% smaller than the radius R₂, for example. The fillet radius R₂ of each engagement tab 11306 is supported by its respective radiused edge 11113. The fillet radius R₂ of each engagement tab 11306 can be fully supported by its respective radiused edge 11113. Such support limits the upward deflection of the engagement tabs 11306 and, as a result, resists the upward movement of the anvil 11100 from its fully-closed position during the staple firing stroke and, thus, reduces the firing force needed to perform the staple firing stroke.

Further to the above, referring again to FIGS. 14, 15 and 17, each engagement tab 11306 comprises a lateral end unconnected to the firing member body 11302. Each lateral end is defined by a curved surface. Each lateral end may be defined by a single radius of curvature. Each lateral end may be defined by more than one radius of curvature. Each lateral end may comprise a spherical shape, for example. Moreover, the anvil 11100 further comprises a radiused corner 11115 extending along each lower ledge 11114 that matches, or at least partially matches, the profile of the lateral end of an engagement tab 11306. Each matched radiused corner 11115 may be defined by a radius that defines a lateral end of an engagement tab 11306. The matched radiused corner 11115 may be defined by a radius that is between 10% larger and 10% smaller than a radius defining a lateral end of an engagement tab 11306, for example. The lateral end of each engagement tab 11306 is supported by its respective radiused corner 11115. Such support limits the upward deflection of the engagement tabs 11306 and, as a result, resists the upward movement of the anvil 11100 from its fully-closed position during the staple firing stroke and, thus, reduces the firing force needed to perform the staple firing stroke.

A surgical stapling instrument can include the anvil closure system of the surgical stapling instrument 1000 and the staple firing system of the surgical stapling instrument 11000. Such a surgical stapling instrument includes the closure tube 1210/1220 that is advanced distally to engage contact ridges 1344 defined on the outside of the anvil and, also, the firing member 11300 that engages the lower ridges 11114 and is supported by the radiused edges 11113 and the radiused corners 11115. Such an arrangement advantageously and co-operatively limits the upward movement of the anvil before and during the staple firing stroke.

Many of the surgical instrument systems described herein are motivated by an electric motor; however, the surgical instrument systems described herein can be motivated in any suitable manner. The surgical instrument systems described herein can be motivated by a manually-operated trigger, for example. The motors disclosed herein may comprise a portion or portions of a robotically controlled system. Moreover, any of the end effectors and/or tool assemblies disclosed herein can be utilized with a robotic surgical instrument system. U.S. patent application Ser. No. 13/118,241, entitled SURGICAL STAPLING INSTRUMENTS WITH ROTATABLE STAPLE DEPLOYMENT ARRANGEMENTS, now U.S. Pat. No. 9,072,535, for example, discloses several examples of a robotic surgical instrument system in greater detail.

The surgical instrument systems described herein have been described in connection with the deployment and deformation of staples; however, the present disclosure may not be so limited. The present disclosure envisions that fasteners other than staples may be deployed, such as clamps or tacks, for example. Moreover, the present disclosure envisions utilizing any suitable means for sealing tissue. An end effector in accordance with the present disclosure can comprise electrodes configured to heat and seal the tissue. Also, an end effector in accordance with the present disclosure can apply vibrational energy to seal the tissue.

The entire disclosures of U.S. Pat. No. 11,589,865, entitled METHODS FOR CONTROLLING A POWERED SURGICAL STAPLER THAT HAS SEPARATE ROTARY CLOSURE AND FIRING SYSTEMS, which issued on Feb. 28, 2023, U.S. Pat. No. 6,978,921, entitled SURGICAL STAPLING INSTRUMENT INCORPORATING AN E-BEAM FIRING MECHANISM, which issued on Dec. 27, 2005, U.S. Pat. No. 10,213,203, entitled STAPLE CARTRIDGE ASSEMBLY WITHOUT A BOTTOM COVER, which issued on Feb. 26, 2019, U.S. Pat. No. 10,945,727, entitled STAPLE CARTRIDGE WITH DEFORMABLE DRIVER RETENTION FEATURES, which issued on Mar. 16, 2021, U.S. Pat. No. 11,234,698, entitled STAPLING SYSTEM COMPRISING A CLAMP LOCKOUT AND A FIRING LOCKOUT, which issued on Feb. 1, 2022, U.S. Pat. No. 11,540,826, entitled SURGICAL STAPLER END EFFECTOR SLED HAVING CARTRIDGE WALL SUPPORT FEATURE, which issued on Jan. 3, 2023, U.S. Pat. No. 10,299,792, entitled FASTENER CARTRIDGE COMPRISING NON-UNIFORM FASTENERS, which issued on May 28, 2019, U.S. Pat. No. 8,540,133, entitled STAPLE CARTRIDGE, which issued on Sep. 24, 2013, U.S. Pat. No. 9,788,835, entitled DEVICES AND METHODS FOR FACILITATING EJECTION OF SURGICAL FASTENERS FROM CARTRIDGES, which issued on Oct. 17, 2017, U.S. Pat. No. 10, 105, 142, entitled SURGICAL STAPLER WITH PLURALITY OF CUTTING ELEMENTS, which issued on Oct. 23, 2018, U.S. Pat. No. 10,537,324, entitled STEPPED STAPLE CARTRIDGE WITH ASYMMETRICAL STAPLES, which issued on Jan. 21, 2020, U.S. Pat. No. 7,669,746, entitled STAPLE CARTRIDGES FOR FORMING STAPLES HAVING DIFFERING FORMED STAPLE HEIGHTS, which issued on Mar. 2, 2010, U.S. Pat. No. 8,123,100, entitled SURGICAL STAPLING INSTRUMENTS INCLUDING A CARTRIDGE HAVING MULTIPLE STAPLE SIZES, which issued on Feb. 28, 2012, U.S. Pat. No. 7,407,075, entitled STAPLE CARTRIDGE HAVING MULTIPLE STAPLE SIZES FOR A SURGICAL STAPLING INSTRUMENT, which issued on Aug. 5, 2008, U.S. Pat. No. 10,085,749, entitled SURGICAL APPARATUS WITH CONDUCTOR STRAIN RELIEF, which issued on Oct. 2, 2018, U.S. Pat. No. 10,765,427, entitled METHOD FOR ARTICULATING A SURGICAL INSTRUMENT, which issued on Sep. 8, 2020, U.S. Pat. No. 11,291,445, entitled SURGICAL STAPLE CARTRIDGES WITH INTEGRAL AUTHENTICATION KEYS, which issued on Apr. 5, 2022, U.S. Pat. No. 8,864,007, entitled IMPLANTABLE FASTENER CARTRIDGE HAVING A NON-UNIFORM ARRANGEMENT, which issued on Oct. 21, 2014, U.S. Pat. No. 11,490,890, entitled COMPRESSIBLE NON-FIBROUS ADJUNCTS, which issued on Nov. 8, 2022, U.S. Pat. No. 10,952,724, entitled THREE DIMENSIONAL ADJUNCTS, which issued on Mar. 23, 2021, U.S. Pat. No. 9,770,245, entitled LAYER ARRANGEMENTS FOR SURGICAL STAPLE CARTRIDGES, which issued on Sep. 26, 2017, U.S. Pat. No. 10,123,798, entitled TISSUE THICKNESS COMPENSATOR COMPRISING CONTROLLED RELEASE AND EXPANSION, which issued on Nov. 13, 2018, U.S. Pat. No. 10,166,023, entitled METHOD OF APPLYING A BUTTRESS TO A SURGICAL STAPLER END EFFECTOR, which issued on Jan. 1, 2019, U.S. Pat. No. 11,207,065, entitled METHOD FOR FABRICATING SURGICAL STAPLER ANVILS, which issued on Dec. 28, 2021, U.S. Pat. No. 8,141,762, entitled SURGICAL STAPLER COMPRISING A STAPLE POCKET, which issued on Mar. 27, 2012, U.S. Pat. No. 8,876,857, entitled END EFFECTOR WITH REDUNDANT CLOSING MECHANISMS, which issued on Nov. 4, 2014, U.S. Pat. No. 9,629,631, entitled COMPOSITE DRIVE BEAM FOR SURGICAL STAPLING, which issued on Apr. 25, 2017, U.S. Patent Application Publication No. 2022/0346858, entitled METHOD FOR OPERATING A SURGICAL INSTRUMENT INCLUDING SEGMENTED ELECTRODES, which published on Nov. 3, 2022, U.S. Patent Application Publication No. 2022/0304680, entitled DRIVERS FOR FASTENER CARTRIDGE ASSEMBLIES HAVING ROTARY DRIVE SCREWS, which published on Sep. 29, 2022, U.S. Patent Application Publication No. 2022/0304679, entitled METHOD OF USING A POWERED STAPLING DEVICE, which published on Sep. 29, 2022, U.S. Patent Publication No. 2019/0298350, entitled METHODS FOR CONTROLLING A POWERED SURGICAL STAPLER THAT HAS SEPARATE ROTARY CLOSURE AND FIRING SYSTEMS, which published on Oct. 3, 2019, U.S. Patent Application Publication No. 2017/0367695, entitled STAPLE CARTRIDGE COMPRISING WIRE STAPLES AND STAMPED STAPLES, which published on Dec. 28, 2017, U.S. Patent Application Publication No. 2015/0134077, entitled SEALING MATERIALS FOR USE IN SURGICAL STAPLING, which published on May 14, 2015, U.S. Patent Application Publication No. 2018/0168615, entitled METHOD OF DEFORMING STAPLES FROM TWO DIFFERENT TYPES OF STAPLE CARTRIDGES WITH THE SAME SURGICAL STAPLING INSTRUMENT, which published on Jun. 21, 2018, U.S. Patent Application Publication No. 2018/0132849, entitled STAPLE FORMING POCKET CONFIGURATIONS FOR CIRCULAR SURGICAL STAPLER ANVIL, which published on May 17, 2018, U.S. Patent Application Publication No. 2018/0168613, entitled SURGICAL INSTRUMENTS WITH JAWS THAT ARE PIVOTABLE ABOUT A FIXED AXIS AND INCLUDE SEPARATE AND DISTINCT CLOSURE AND FIRING SYSTEMS, which published on Jun. 21, 2018, U.S. Patent Application Publication No. 2017/0319205, entitled POWERED END EFFECTOR ASSEMBLY WITH PIVOTABLE CHANNEL, which published on Nov. 9, 2017, U.S. Patent Application Publication No. 2014/0001231, entitled FIRING SYSTEM LOCKOUT ARRANGEMENTS FOR SURGICAL INSTRUMENTS, which published on Jan. 2, 2014, U.S. Patent Application Publication No. 2016/0095596, entitled APPARATUS FOR ENDOSCOPIC PROCEDURES, which published on Apr. 7, 2016, U.S. Patent Application Publication No. 2015/0297199, entitled ADAPTER ASSEMBLY WITH GIMBAL FOR INTERCONNECTING ELECTROMECHANICAL SURGICAL DEVICES AND SURGICAL LOADING UNITS, AND SURGICAL SYSTEMS THEREOF, which published on Oct. 22, 2015, U.S. Patent Application Publication No. 2022/0031351, entitled SURGICAL INSTRUMENTS WITH DIFFERENT ARTICULATION JOINT ARRANGEMENTS FOR ACCOMMODATING FLEXIBLE ACTUATORS, which published on Feb. 3, 2022, U.S. Patent Application Publication No. 2022/0031320, entitled SURGICAL INSTRUMENTS WITH FLEXIBLE FIRING MEMBER ACTUATOR CONSTRAINT ARRANGEMENTS, which published on Feb. 3, 2022, U.S. Patent Application Publication No. 2023/0119119, entitled CABLE-DRIVEN ACTUATION SYSTEM FOR ROBOTIC SURGICAL TOOL ATTACHMENT, which published on Apr. 20, 2023, International Patent Publication No. WO2018/071497, entitled STAPLER CARTRIDGE WITH AN INTEGRAL KNIFE, which published on Apr. 18, 2018, International Patent Publication No. WO2018/049211, entitled WRIST ARCHITECTURE, which published on Mar. 15, 2018, U.S. Pat. No. 11,298,129, entitled METHOD FOR PROVIDING AN AUTHENTICATION LOCKOUT IN A SURGICAL STAPLER WITH A REPLACEABLE CARTRIDGE, which issued on Apr. 12, 2022, U.S. Pat. No. 10,898,183, entitled ROBOTIC SURGICAL INSTRUMENT WITH CLOSED LOOP FEEDBACK TECHNIQUES FOR ADVANCEMENT OF CLOSURE MEMBER DURING FIRING, which issued on Jan. 26, 2021, U.S. Pat. No. 5,485,947, entitled LINEAR STAPLING MECHANISM WITH CUTTING MEANS, which issued on Jan. 23, 1996, International Patent Publication No. WO2018/049206, entitled STAPLER RELOAD DETECTION AND IDENTIFICATION, which published on Mar. 15, 2018, U.S. Patent Application Publication No. 2016/0249920, entitled Surgical fastener applying apparatus, which published on Sep. 1, 2016, U.S. Design Pat. No. D974,560, entitled STAPLE CARTRIDGE, which issued on Jan. 3, 2023, U.S. Design Pat. No. D967,421, entitled STAPLE CARTRIDGE, which issued on Oct. 18, 2022, U.S. Design Pat. No. D933,220, entitled BUTTRESS ASSEMBLY FOR A SURGICAL STAPLER, which issued on Oct. 12, 2021, U.S. Pat. No. 9,839,420, entitled TISSUE THICKNESS COMPENSATOR COMPRISING AT LEAST ONE MEDICAMENT, which issued on Dec. 12, 2017, U.S. Pat. No. 10,588,623, entitled ADHESIVE FILM LAMINATE, which issued on Mar. 17, 2020, U.S. Pat. No. 8,499,992, entitled DEVICE AND METHOD FOR CONTROLLING COMPRESSION OF TISSUE, which issued on Aug. 6, 2013, U.S. Patent Application Publication No. 2022/0378427, entitled STAPLING INSTRUMENT COMPRISING JAW MOUNTS, which published on Dec. 1, 2022, U.S. Pat. No. 10,349,939, entitled METHOD OF APPLYING A BUTTRESS TO A SURGICAL STAPLER, which issued on Jul. 16, 2019, U.S. Pat. No. 9,386,988, entitled RETAINER ASSEMBLY INCLUDING A TISSUE THICKNESS COMPENSATOR, which issued on Jul. 12, 2016, U.S. Pat. No. 9,072,535, entitled SURGICAL STAPLING INSTRUMENTS WITH ROTATABLE STAPLE DEPLOYMENT ARRANGEMENTS, which issued on Jul. 7, 2015, and U.S. Pat. No. 9,844,369, entitled, SURGICAL END EFFECTORS WITH FIRING ELEMENT MONITORING ARRANGEMENTS, which issued on Dec. 19, 2017 are incorporated by reference herein.

The entire disclosures of:
U.S. Pat. No. 5,403,312, entitled ELECTROSURGICAL HEMOSTATIC DEVICE, which issued on Apr. 4, 1995;
U.S. Pat. No. 7,000,818, entitled SURGICAL STAPLING INSTRUMENT HAVING SEPARATE DISTINCT CLOSING AND FIRING SYSTEMS, which issued on Feb. 21, 2006;
U.S. Pat. No. 7,422,139, entitled MOTOR-DRIVEN SURGICAL CUTTING AND FASTENING INSTRUMENT WITH TACTILE POSITION FEEDBACK, which issued on Sep. 9, 2008;
U.S. Pat. No. 7,464,849, entitled ELECTRO-MECHANICAL SURGICAL INSTRUMENT WITH CLOSURE SYSTEM AND ANVIL ALIGNMENT COMPONENTS, which issued on Dec. 16, 2008;
U.S. Pat. No. 7,670,334, entitled SURGICAL INSTRUMENT HAVING AN ARTICULATING END EFFECTOR, which issued on Mar. 2, 2010;
U.S. Pat. No. 7,753,245, entitled SURGICAL STAPLING INSTRUMENTS, which issued on Jul. 13, 2010;
U.S. Pat. No. 8,393,514, entitled SELECTIVELY ORIENTABLE IMPLANTABLE FASTENER CARTRIDGE, which issued on Mar. 12, 2013;
U.S. patent application Ser. No. 11/343,803, entitled SURGICAL INSTRUMENT HAVING RECORDING CAPABILITIES, now U.S. Pat. No. 7,845,537;
U.S. patent application Ser. No. 12/031,573, entitled SURGICAL CUTTING AND FASTENING INSTRUMENT HAVING RF ELECTRODES, filed Feb. 14, 2008;
U.S. patent application Ser. No. 12/031,873, entitled END EFFECTORS FOR A SURGICAL CUTTING AND STAPLING INSTRUMENT, filed Feb. 15, 2008, now U.S. Pat. No. 7,980,443;
U.S. patent application Ser. No. 12/235,782, entitled MOTOR-DRIVEN SURGICAL CUTTING INSTRUMENT, now U.S. Pat. No. 8,210,411;
U.S. patent application Ser. No. 12/235,972, entitled MOTORIZED SURGICAL INSTRUMENT, now U.S. Pat. No. 9,050,083.
U.S. patent application Ser. No. 12/249,117, entitled POWERED SURGICAL CUTTING AND STAPLING APPARATUS WITH MANUALLY RETRACTABLE FIRING SYSTEM, now U.S. Pat. No. 8,608,045;
U.S. patent application Ser. No. 12/647,100, entitled MOTOR-DRIVEN SURGICAL CUTTING INSTRUMENT WITH ELECTRIC ACTUATOR DIRECTIONAL CONTROL ASSEMBLY, filed Dec. 24, 2009, now U.S. Pat. No. 8,220,688;
U.S. patent application Ser. No. 12/893,461, entitled STAPLE CARTRIDGE, filed Sep. 29, 2012, now U.S. Pat. No. 8,733,613;
U.S. patent application Ser. No. 13/036,647, entitled SURGICAL STAPLING INSTRUMENT, filed Feb. 28, 2011, now U.S. Pat. No. 8,561,870;
U.S. patent application Ser. No. 13/118,241, entitled SURGICAL STAPLING INSTRUMENTS WITH ROTATABLE STAPLE DEPLOYMENT ARRANGEMENTS, now U.S. Pat. No. 9,072,535;
U.S. patent application Ser. No. 13/524,049, entitled ARTICULATABLE SURGICAL INSTRUMENT COMPRISING A FIRING DRIVE, filed on Jun. 15, 2012, now U.S. Pat. No. 9,101,358;
U.S. patent application Ser. No. 13/800,025, entitled STAPLE CARTRIDGE TISSUE THICKNESS SENSOR SYSTEM, filed on Mar. 13, 2013, now U.S. Pat. No. 9,345,481;
U.S. patent application Ser. No. 13/800,067, entitled STAPLE CARTRIDGE TISSUE THICKNESS SENSOR SYSTEM, filed on Mar. 13, 2013, now U.S. Patent Application Publication No. 2014/0263552;
U.S. Patent Application Publication No. 2007/0175955, entitled SURGICAL CUTTING AND FASTENING INSTRUMENT WITH CLOSURE TRIGGER LOCKING MECHANISM, filed Jan. 31, 2006; and
U.S. Patent Application Publication No. 2010/0264194, entitled SURGICAL STAPLING INSTRUMENT WITH AN ARTICULATABLE END EFFECTOR, filed Apr. 22, 2010, now U.S. Pat. No. 8,308,040, are hereby incorporated by reference herein.

Various aspects of the subject matter described herein are set out in the following examples.

1. A surgical instrument assembly comprising a first jaw and a second jaw rotatably coupled to said first jaw, wherein said second jaw is movable between an open position and a closed position, and wherein said second jaw comprises a proximal end, a distal end, a longitudinal jaw axis extending between said proximal end and said distal end, a first circumferential periphery extending about said longitudinal jaw axis, wherein said first circumferential periphery comprises first contact ridges, and a second circumferential periphery extending about said longitudinal jaw axis, wherein said second circumferential periphery comprises second ridges and clearance flats, wherein each said clearance flat is positioned intermediate two said second ridges, and wherein said first circumferential periphery is situated distally with respect to said second circumferential periphery. The surgical instrument assembly further comprises a closure tube movable from a proximal position to a distal position during a closure stroke, wherein said closure tube is configured to contact said first contact ridges during said closure stroke to move said second jaw into said closed position, wherein said closure tube is movable proximally from said distal position toward said proximal position to permit said second jaw to return to said open position, and wherein said second ridges are in contact with said closure tube when said second jaw is in said open position.
2. The surgical instrument assembly of Example 1, wherein said first circumferential periphery comprises first flat surfaces, and wherein each said first flat surface is positioned intermediate two said first contact ridges.
3. The surgical instrument assembly of Example 1 or 2, wherein said first contact ridges are arranged in an array, wherein said array of first contact ridges comprises a first segment on a first side of said longitudinal jaw axis and a second segment on a second side of said longitudinal jaw axis, and wherein said first segment is separated from said second segment.
4. The surgical instrument assembly of Example 1, 2, or 3, wherein said closure tube further comprises a first jaw opening tab and a second jaw opening tab, wherein said first jaw opening tab is positioned longitudinally with respect to said second jaw opening tab, wherein said first jaw opening tab is configured to engage said second jaw when said closure tube is moved proximally during a first opening motion to move said second jaw toward said open position, wherein said second jaw opening tab is configured to not engage said second jaw during said first opening motion, and wherein said second jaw opening tab is configured to engage said second jaw during a second opening motion that is subsequent to said first opening motion to move said second jaw into said open position.
5. The surgical instrument assembly of Example 1, 2, 3, or 4, wherein each said clearance flat is aligned with a said first contact ridge.
6. The surgical instrument assembly of Example 1 or 2, wherein said second ridges are arranged in an array, wherein said array comprises a segment on a first side of said longitudinal jaw axis and a second segment on a second side of said longitudinal jaw axis, and wherein said first segment is separated from said second segment.
7. The surgical instrument assembly of Example 1, 2, 3, 4, 5, or 6, wherein said first contact ridges are longitudinally registered with said clearance flats.
8. The surgical instrument assembly of Example 1, 2, 3, 4, 5, 6, or 7, wherein said first contact ridges are longitudinally out of register with said second ridges.
9. The surgical instrument assembly of Example 1, 2, 3, 4, 5, 6, 7, or 8, wherein said second jaw further comprises a cam ramp situated proximally with respect to said first contact ridges and situated distally with respect to said second ridges, wherein said closure tube is configured to contact said cam ramp before contacting said first ridges during said closure stroke, wherein said closure tube is configured to contact said camp ram to move said second jaw from said open position toward a partially closed position during said closure stroke, and wherein said closure tube is configured to contact said first contact ridges to move said second jaw from said partially closed position into said closed position during said closure stroke.
10. The surgical instrument assembly of Example 9, wherein said cam ramp connects said first circumferential periphery and said second circumferential periphery.
11. The surgical instrument assembly of Example 1, 2, 3, 4, 5, 6, 7, 8, 9, or 10, wherein said first jaw comprises a channel configured to receive a replaceable staple cartridge comprising staples removably stored therein and said second jaw comprises an anvil, and wherein said anvil comprises staple forming pockets.
12. The surgical instrument assembly of Example 11, further comprising said replaceable staple cartridge.
13. The surgical instrument assembly of Example 1, 2, 3, 4, 5, 6, 7, 8, 9, 10, 11, or 12, wherein said closure tube further comprises a first jaw opening tab and a second jaw opening tab, wherein said first jaw opening tab is positioned on a first lateral side of said longitudinal jaw axis and said second jaw opening tab is positioned on a second lateral side of said longitudinal jaw axis, wherein said first jaw opening tab is configured to engage said second jaw when said closure tube is moved proximally during a first opening motion to move said second jaw toward said open position, wherein said second jaw opening tab is configured to not engage said second jaw during said first opening motion, and wherein said second jaw opening tab is configured to engage said second jaw during a second opening motion that is subsequent to said first opening motion to move said second jaw into said open position.
14. The surgical instrument assembly of Example 13, wherein said first jaw opening tab is positioned distally with respect to said second jaw opening tab.
15. The surgical instrument assembly of Example 1, 2, 3, 4, 5, 6, 7, 8, 9, 10, 11, 12, 13, or 14, wherein said second jaw is rotatable along an opening axis, wherein said first contact ridges comprise a first lateral control edge and a second lateral control edge, wherein said first lateral control edge and said second lateral control edge define an axis that is orthogonal to said opening axis, and wherein said closure tube engages said first lateral control edge and said second lateral control edge during said closure stroke to limit distortion of said closure tube during the closure stroke.
16. A surgical instrument assembly comprising a first jaw and a second jaw rotatably coupled to said first jaw, wherein said second jaw is movable between an open position and a fully clamped position, and wherein said second jaw comprises a proximal end, a distal end, a longitudinal jaw axis extending between said proximal end and said distal end, a first periphery extending about said longitudinal jaw axis, wherein said first periphery comprises first control surfaces, and a second periphery extending about said longitudinal jaw axis, wherein said second periphery comprises second control surfaces, and wherein said first periphery is situated distally with respect to said second periphery. The surgical instrument assembly further comprising a clamping driver movable from a proximal position to a distal position during a clamping stroke, wherein said clamping driver is configured to contact said first control surfaces during said clamping stroke to move said second jaw into said fully clamped position, wherein said closure tube is movable proximally from said distal position toward said proximal position to permit said second jaw to return to said open position, and wherein said second control surfaces are in contact with said clamping driver when said second jaw is in said open position.
17. A surgical instrument assembly comprising a first jaw and a second jaw rotatably coupled to said first jaw, wherein said second jaw is movable between an open position and a fully-clamped position along an opening axis, and wherein said second jaw comprises a proximal end, a distal end, a longitudinal jaw axis extending between said proximal end and said distal end, and a periphery extending about said longitudinal jaw axis, wherein said periphery comprises a first lateral control edge and a second lateral control edge, wherein said first lateral control edge and said second lateral control edge define an outer control dimension of said second jaw. The surgical instrument further comprises a closure tube movable from a proximal position to a distal position during a clamping stroke to move said second jaw into said fully-clamped position, wherein said closure tube comprises an inner sidewall that defines in an inner longitudinal aperture, and wherein said inner sidewall engages said first lateral control edge and said second lateral control edge during said closure stroke to limit distortion of said closure tube during the clamping stroke.
18. The surgical instrument assembly of Example 17, wherein said first lateral control edge and said second lateral control edge define an axis that is orthogonal to said opening axis.
19. The surgical instrument assembly of Example 17 or 18, further comprising a firing driver movable through a staple firing stroke, wherein contact between said inner sidewall and said first lateral control edge and said second lateral control edge limit the distortion of said closure tube during the staple firing stroke.
20. The surgical instrument assembly of Example 19, wherein said second jaw comprises a longitudinal channel defined therein, wherein said firing driver comprises a firing driver body, a first cam extending from said firing driver body configured to engage said first jaw during the staple firing stroke, and a second cam extending from said firing driver body configured to engage said second jaw during the staple firing stroke, wherein said second cam comprises a base connected to said firing driver body, a fillet at said base, and a round lateral end, and wherein said longitudinal channel comprises a longitudinal ledge comprising a radiused edge defined by a radius of curvature that matches a radius of curvature of said fillet, wherein said second cam is configured to slide along said longitudinal ledge during the staple firing stroke, a longitudinal wall extending alongside said longitudinal ledge, and a longitudinal corner defined between said longitudinal ledge and said longitudinal wall, wherein said longitudinal corner is defined by a curvature that matches said round lateral end.
21. The surgical instrument assembly of Example 17, 18, 19, or 20, wherein said first lateral control edge and said second lateral control edge extend longitudinally when said second jaw is in said fully-clamped position.

Various aspects of the subject matter described herein are set out in the following additional examples.

1. A surgical instrument assembly comprising a first jaw (1310), a second jaw (1320) rotatably coupled to said first jaw, and a closure tube (1210) movable from a proximal position to a distal position during a closure stroke. The second jaw is movable between an open position and a closed position. The second jaw comprises a proximal end (1328), a distal end (1329), a longitudinal jaw axis (JA) extending between said proximal end and said distal end, a first circumferential periphery (1340) extending about said longitudinal jaw axis, a second circumferential periphery (1330) extending about said longitudinal jaw axis. The first circumferential periphery comprises first contact ridges (1344). The second circumferential periphery comprises second ridges (1334) and clearance flats (1332). Each said clearance flat is positioned intermediate two said second ridges. The first circumferential periphery is situated distally with respect to said second circumferential periphery. The closure tube is configured to contact said first contact ridges during said closure stroke to move said second jaw into said closed position. The closure tube is movable proximally from said distal position toward said proximal position to permit said second jaw to return to said open position. The second ridges are in contact with said closure tube when said second jaw is in said open position.
2. The surgical instrument assembly of Example 1, wherein said first circumferential periphery (1340) comprises first flat surfaces (1342). Each said first flat surface is positioned intermediate two said first contact ridges (1344).
3. The surgical instrument assembly of Examples 1 or 2, wherein said first contact ridges (1344) are arranged in an array. The array of first contact ridges comprises a first segment on a first side of said longitudinal jaw axis (JA) and a second segment on a second side of said longitudinal jaw axis. The first segment is separated from said second segment.
4. The surgical instrument assembly of Examples 1, 2, or 3, wherein said closure tube (1210) further comprises a first jaw opening tab (1229) and a second jaw opening tab (1229). The first jaw opening tab is positioned longitudinally with respect to said second jaw opening tab. The first jaw opening tab is configured to engage said second jaw (1320) when said closure tube is moved proximally during a first opening motion to move said second jaw toward said open position. The second jaw opening tab is configured to not engage said second jaw during said first opening motion. The second jaw opening tab is configured to engage said second jaw during a second opening motion that is subsequent to said first opening motion to move said second jaw into said open position.
5. The surgical instrument assembly of Examples 1, 2, 3, or 4, wherein each said clearance flat (1332) is aligned with a said first contact ridge (1344).
6. The surgical instrument assembly of Examples 1 or 2, wherein said second ridges (1334) are arranged in an array. The array comprises a segment on a first side of said longitudinal jaw axis (JA) and a second segment on a second side of said longitudinal jaw axis. The first segment is separated from said second segment.
7. The surgical instrument assembly of Examples 1, 2, 3, 4, 5, or 6, wherein said first contact ridges (1344) are longitudinally registered with said clearance flats (1332).
8. The surgical instrument assembly of Examples 1, 2, 3, 4, 5, 6, or 7, wherein said first contact ridges (1344) are longitudinally out of register with said second ridges (1334).
9. The surgical instrument assembly of Examples 1, 2, 3, 4, 5, 6, 7, or 8, wherein said second jaw (1320) further comprises a cam ramp (1327) situated proximally with respect to said first contact ridges (1344) and situated distally with respect to said second ridges (1334). The closure tube (1210) is configured to contact said cam ramp before contacting said first ridges during said closure stroke. The closure tube is configured to contact said camp ram to move said second jaw from said open position toward a partially closed position during said closure stroke. The closure tube is configured to contact said first contact ridges to move said second jaw from said partially closed position into said closed position during said closure stroke.
10. The surgical instrument assembly of Example 9, wherein said cam ramp (1327) connects said first circumferential periphery (1340) and said second circumferential periphery (1330).
11. The surgical instrument assembly of Examples 1, 2, 3, 4, 5, 6, 7, 8, 9, or 10, wherein said first jaw (1310) comprises a channel (1314) configured to receive a replaceable staple cartridge (1500) comprising staples removably stored therein and said second jaw (1320) comprises an anvil. The anvil comprises staple forming pockets.
12. The surgical instrument assembly of Example 11, further comprising said replaceable staple cartridge (1500).
13. The surgical instrument assembly of Examples 1, 2, 3, 4, 5, 6, 7, 8, 9, 10, 11, or 12, wherein said closure tube (1210) further comprises a first jaw opening tab (1229) and a second jaw opening tab (1229). The first jaw opening tab is positioned on a first lateral side of said longitudinal jaw axis (JA) and said second jaw opening tab is positioned on a second lateral side of said longitudinal jaw axis. The first jaw opening tab is configured to engage said second jaw when said closure tube is moved proximally during a first opening motion to move said second jaw toward said open position. The second jaw opening tab is configured to not engage said second jaw during said first opening motion. The second jaw opening tab is configured to engage said second jaw during a second opening motion that is subsequent to said first opening motion to move said second jaw into said open position.
14. The surgical instrument assembly of Example 13, wherein said first jaw opening tab is positioned distally with respect to said second jaw opening tab.
15. The surgical instrument assembly of Examples 1, 2, 3, 4, 5, 6, 7, 8, 9, 10, 11, 12, 13, or 14, wherein said second jaw is rotatable along an opening axis (12-6A). The first contact ridges comprise a first lateral control edge and a second lateral control edge. The first lateral control edge and said second lateral control edge define an axis (3-9A) that is orthogonal to said opening axis. The closure tube engages said first lateral control edge and said second lateral control edge during said closure stroke to limit distortion of said closure tube during the closure stroke.

Although various devices have been described herein in connection with certain embodiments, modifications and variations to those embodiments may be implemented. Particular features, structures, or characteristics may be combined in any suitable manner in one or more embodiments. Thus, the particular features, structures, or characteristics illustrated or described in connection with one embodiment may be combined in whole or in part, with the features, structures or characteristics of one ore more other embodiments without limitation. Also, where materials are disclosed for certain components, other materials may be used. Furthermore, according to various embodiments, a single component may be replaced by multiple components, and multiple components may be replaced by a single component, to perform a given function or functions. The foregoing description and following claims are intended to cover all such modification and variations.

The devices disclosed herein can be designed to be disposed of after a single use, or they can be designed to be used multiple times. In either case, however, a device can be reconditioned for reuse after at least one use. Reconditioning can include any combination of the steps including, but not limited to, the disassembly of the device, followed by cleaning or replacement of particular pieces of the device, and subsequent reassembly of the device. In particular, a reconditioning facility and/or surgical team can disassemble a device and, after cleaning and/or replacing particular parts of the device, the device can be reassembled for subsequent use. Those skilled in the art will appreciate that reconditioning of a device can utilize a variety of techniques for disassembly, cleaning/replacement, and reassembly. Use of such techniques, and the resulting reconditioned device, are all within the scope of the present application.

The devices disclosed herein may be processed before surgery. First, a new or used instrument may be obtained and, when necessary, cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, and/or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta radiation, gamma radiation, ethylene oxide, plasma peroxide, and/or steam.

While this invention has been described as having exemplary designs, the present invention may be further modified within the spirit and scope of the disclosure. This application is therefore intended to cover any variations, uses, or adaptations of the invention using its general principles.

It is worthy to note that any reference numbers included in the appended claims are used to reference exemplary embodiments/elements described in the present disclosure. Accordingly, any such reference numbers are not meant to limit the scope of the subject matter recited in the appended claims.

1-15. (canceled)

16. A surgical instrument assembly, comprising:
a first jaw;
a second jaw rotatably coupled to said first jaw, wherein said second jaw is movable between an open position and a closed position, and wherein said second jaw comprises:
a proximal end;
a distal end;
a longitudinal jaw axis extending between said proximal end and said distal end;
a first circumferential periphery extending about said longitudinal jaw axis, wherein said first circumferential periphery comprises first contact ridges; and
a second circumferential periphery extending about said longitudinal jaw axis, wherein said second circumferential periphery comprises second ridges and clearance flats, wherein each said clearance flat is positioned intermediate two said second ridges, and wherein said first circumferential periphery is situated distally with respect to said second circumferential periphery; and
a closure tube movable from a proximal position to a distal position during a closure stroke, wherein said closure tube is configured to contact said first contact ridges during said closure stroke to move said second jaw into said closed position, wherein said closure tube is movable proximally from said distal position toward said proximal position to permit said second jaw to return to said open position, and wherein said second ridges are in contact with said closure tube when said second jaw is in said open position.

17. The surgical instrument assembly of claim 16, wherein said first circumferential periphery comprises first flat surfaces, and wherein each said first flat surface is positioned intermediate two said first contact ridges.

18. The surgical instrument assembly of claim 16, wherein said first contact ridges are arranged in an array, wherein said array of first contact ridges comprises a first segment on a first side of said longitudinal jaw axis (JA) and a second segment on a second side of said longitudinal jaw axis, and wherein said first segment is separated from said second segment.

19. The surgical instrument assembly of claim 16, wherein said closure tube further comprises a first jaw opening tab and a second jaw opening tab, wherein said first jaw opening tab is positioned longitudinally with respect to said second jaw opening tab, wherein said first jaw opening tab is configured to engage said second jaw when said closure tube is moved proximally during a first opening motion to move said second jaw toward said open position, wherein said second jaw opening tab is configured to not engage said second jaw during said first opening motion, and wherein said second jaw opening tab is configured to engage said second jaw during a second opening motion that is subsequent to said first opening motion to move said second jaw into said open position.

20. The surgical instrument assembly of claim 16, wherein each said clearance flat is aligned with a said first contact ridge.

21. The surgical instrument assembly of claim 16, wherein said second ridges are arranged in an array, wherein said array comprises a segment on a first side of said longitudinal jaw axis and a second segment on a second side of said longitudinal jaw axis, and wherein said first segment is separated from said second segment.

22. The surgical instrument assembly of claim 16, wherein said first contact ridges are longitudinally registered with said clearance flats.

23. The surgical instrument assembly of claim 16, wherein said first contact ridges are longitudinally out of register with said second ridges.

24. The surgical instrument assembly of claim 16, wherein said second jaw further comprises a cam ramp situated proximally with respect to said first contact ridges and situated distally with respect to said second ridges, wherein said closure tube is configured to contact said cam ramp before contacting said first ridges during said closure stroke, wherein said closure tube is configured to contact said camp ram to move said second jaw from said open position toward a partially closed position during said closure stroke, and wherein said closure tube is configured to contact said first contact ridges to move said second jaw from said partially closed position into said closed position during said closure stroke.

25. The surgical instrument assembly of claim 24, wherein said cam ramp connects said first circumferential periphery and said second circumferential periphery.

26. The surgical instrument assembly of claim 16, wherein said first jaw comprises a channel configured to receive a replaceable staple cartridge comprising staples removably stored therein and said second jaw comprises an anvil, and wherein said anvil comprises staple forming pockets.

27. The surgical instrument assembly of claim 26, further comprising said replaceable staple cartridge.

28. The surgical instrument assembly of claim 16, wherein said closure tube further comprises a first jaw opening tab and a second jaw opening tab, wherein said first jaw opening tab is positioned on a first lateral side of said longitudinal jaw axis and said second jaw opening tab is positioned on a second lateral side of said longitudinal jaw axis, wherein said first jaw opening tab is configured to engage said second jaw when said closure tube is moved proximally during a first opening motion to move said second jaw toward said open position, wherein said second jaw opening tab is configured to not engage said second jaw during said first opening motion, and wherein said second jaw opening tab is configured to engage said second jaw during a second opening motion that is subsequent to said first opening motion to move said second jaw into said open position.

29. The surgical instrument assembly of claim 28, wherein said first jaw opening tab is positioned distally with respect to said second jaw opening tab.

30. The surgical instrument assembly of claim 16, wherein said second jaw is rotatable along an opening axis, wherein said first contact ridges comprise a first lateral control edge and a second lateral control edge, wherein said first lateral control edge and said second lateral control edge define an axis that is orthogonal to said opening axis, and wherein said closure tube engages said first lateral control edge and said second lateral control edge during said closure stroke to limit distortion of said closure tube during the closure stroke.

31. A surgical instrument assembly, comprising:
a first jaw;
a second jaw rotatably coupled to said first jaw, wherein said second jaw is movable between an open position and a fully clamped position, and wherein said second jaw comprises:
a proximal end;
a distal end;
a longitudinal jaw axis extending between said proximal end and said distal end;
a first periphery extending about said longitudinal jaw axis, wherein said first periphery comprises first control surfaces; and
a second periphery extending about said longitudinal jaw axis, wherein said second periphery comprises second control surfaces, and wherein said first periphery is situated distally with respect to said second periphery; and
a clamping driver movable from a proximal position to a distal position during a clamping stroke, wherein said clamping driver is configured to contact said first control surfaces during said clamping stroke to move said second jaw into said fully clamped position, wherein said closure tube is movable proximally from said distal position toward said proximal position to permit said second jaw to return to said open position, and wherein said second control surfaces are in contact with said clamping driver when said second jaw is in said open position.

32. A surgical instrument assembly, comprising:
a first jaw;
a second jaw rotatably coupled to said first jaw, wherein said second jaw is movable between an open position and a fully-clamped position along an opening axis, and wherein said second jaw comprises:
a proximal end;
a distal end;
a longitudinal jaw axis extending between said proximal end and said distal end; and
a periphery extending about said longitudinal jaw axis, wherein said periphery comprises a first lateral control edge and a second lateral control edge, wherein said first lateral control edge and said second lateral control edge define an outer control dimension of said second jaw; and
a closure tube movable from a proximal position to a distal position during a clamping stroke to move said second jaw into said fully-clamped position, wherein said closure tube comprises an inner sidewall that defines in an inner longitudinal aperture, and wherein said inner sidewall engages said first lateral control edge and said second lateral control edge during said closure stroke to limit distortion of said closure tube during the clamping stroke.

33. The surgical instrument assembly of claim 32, wherein said first lateral control edge and said second lateral control edge define an axis that is orthogonal to said opening axis.

34. The surgical instrument assembly of claim 32, further comprising a firing driver movable through a staple firing stroke, wherein contact between said inner sidewall and said first lateral control edge and said second lateral control edge limit the distortion of said closure tube during the staple firing stroke.

35. The surgical instrument assembly of claim 34, wherein said second jaw comprises a longitudinal channel defined therein, wherein said firing driver comprises a firing driver body, a first cam extending from said firing driver body configured to engage said first jaw during the staple firing stroke, and a second cam extending from said firing driver body configured to engage said second jaw during the staple firing stroke, wherein said second cam comprises a base connected to said firing driver body, a fillet at said base, and a round lateral end, and wherein said longitudinal channel comprises:
a longitudinal ledge comprising a radiused edge defined by a radius of curvature that matches a radius of curvature of said fillet, wherein said second cam is configured to slide along said longitudinal ledge during the staple firing stroke;
a longitudinal wall extending alongside said longitudinal ledge; and
a longitudinal corner defined between said longitudinal ledge and said longitudinal wall, wherein said longitudinal corner is defined by a curvature that matches said round lateral end.

36. The surgical instrument assembly of claim 32, wherein said first lateral control edge and said second lateral control edge extend longitudinally when said second jaw is in said fully-clamped position.
